SEQUENCE LISTING (216 sequences)

SEQ ID NO: 1 - DNA, 19 bp, Artificial Sequence
  Proteo-1 Oligonucleotide
gacaagatggtgtcctgag

SEQ ID NO: 2 - DNA, 17 bp, Artificial Sequence
  Proteo-2 Oligonucleotide
gacaggatggtgtcctg

SEQ ID NO: 3 - DNA, 17 bp, Artificial Sequence
  Proteo-3 Oligonucleotide
ggctcaggatggtgtcc

SEQ ID NO: 4 - DNA, 16 bp, Artificial Sequence
  Proteo-4 Oligonucleotide
aggacggtgtcctggg

SEQ ID NO: 5 - DNA, 17 bp, Artificial Sequence
  Proteo-5 Oligonucleotide
ttccgcggcaagggaga

SEQ ID NO: 6 - DNA, 17 bp, Artificial Sequence
  Proteo-6 Oligonucleotide
gtgtcctgcagcttggt

SEQ ID NO: 7 - DNA, 19 bp, Artificial Sequence
  Proteo-7 Oligonucleotide
agccgatcttgagaacgtc

SEQ ID NO: 8 - DNA, 18 bp, Artificial Sequence
  Proteo-8 Oligonucleotide
agccgatctgcagcacgt

SEQ ID NO: 9 - DNA, 19 bp, Artificial Sequence
  Proteo-9 Oligonucleotide
cagttccatcacggagttc

SEQ ID NO: 10 - DNA, 18 bp, Artificial Sequence
  Proteo-10 Oligonucleotide
tgcatgacgctggtttgc

SEQ ID NO: 11 - DNA, 19 bp, Artificial Sequence
  Proteo-11 Oligonucleotide
tacccgatggccagcacat

SEQ ID NO: 12 - DNA, 20 bp, Artificial Sequence
  Proteo-12 Oligonucleotide
atgacggtgttctgcatctt

SEQ ID NO: 13 - DNA, 18 bp, Artificial Sequence
  Proteo-13 Oligonucleotide
cacggtcgtttgcgcctt

SEQ ID NO: 14 - DNA, 19 bp, Artificial Sequence
  Proteo-14 Oligonucleotide
tctgagcctttgagtgcag

SEQ ID NO: 15 - DNA, 18 bp, Artificial Sequence
  Proteo-15 Oligonucleotide
ttgatgtcgccgtagccg

SEQ ID NO: 16 - DNA, 21 bp, Artificial Sequence
  Proteo-16 Oligonucleotide
tttaatgccgctgtaaccagt

SEQ ID NO: 17 - DNA, 22 bp, Artificial Sequence
  Proteo-17 Oligonucleotide
ccagtcagtagtacatcttcta

SEQ ID NO: 18 - DNA, 20 bp, Artificial Sequence
  Proteo-18 Oligonucleotide
ttttgcgctttcgcatgcag

SEQ ID NO: 19 - DNA, 18 bp, Artificial Sequence
  Proteo-19 Oligonucleotide
cttgctgtggaggatcag

SEQ ID NO: 20 - DNA, 21 bp, Artificial Sequence
  Proteo-20 Oligonucleotide
ctccattattgtgttttgcgc

SEQ ID NO: 21 - DNA, 20 bp, Artificial Sequence
  Proteo-21 Oligonucleotide
gtgttttgcgctttagagtg

SEQ ID NO: 22 - DNA, 22 bp, Artificial Sequence
  Proteo-22 Oligonucleotide
ggaagttaatcgctgtgataac

SEQ ID NO: 23 - DNA, 17 bp, Artificial Sequence
  Proteo-23 Oligonucleotide
atggtctcctgggcctt

SEQ ID NO: 24 - DNA, 17 bp, Artificial Sequence
  Proteo-24 Oligonucleotide
cacttgacgtcgccgta

SEQ ID NO: 25 - DNA, 21 bp, Artificial Sequence
  Proteo-25 Oligonucleotide
ttccaaatcttcaacgctacc

SEQ ID NO: 26 - DNA, 17 bp, Artificial Sequence
  Proteo-26 Oligonucleotide
gtgacagcaccgacgtc

SEQ ID NO: 27 - DNA, 17 bp, Artificial Sequence
  Proteo-27 Oligonucleotide
acggtctgctgggcttt

SEQ ID NO: 28 - DNA, 18 bp, Artificial Sequence
  Proteo-28 Oligonucleotide
atgcaggaccgtgtcctg

SEQ ID NO: 29 - DNA, 19 bp, Artificial Sequence
  Proteo-29 Oligonucleotide
agatgcagaaccgtgtcct

SEQ ID NO: 30 - DNA, 17 bp, Artificial Sequence
  Proteo-30 Oligonucleotide
gaaccttcggttgccgc

SEQ ID NO: 31 - DNA, 17 bp, Artificial Sequence
  Proteo-31 Oligonucleotide
gcggcgagatgcagaac

SEQ ID NO: 32 - DNA, 18 bp, Artificial Sequence
  Proteo-32 Oligonucleotide
atccaggacggtatcctg

SEQ ID NO: 33 - DNA, 18 bp, Artificial Sequence
  Proteo-33 Oligonucleotide
gatgtcctgatagccgac

SEQ ID NO: 34 - DNA, 19 bp, Artificial Sequence
  Proteo-34 Oligonucleotide
ttgatgtccttgaagccga

SEQ ID NO: 35 - DNA, 21 bp, Artificial Sequence
  Proteo-35 Oligonucleotide
gcctgattcaacacaacgaac

SEQ ID NO: 36 - DNA, 17 bp, Artificial Sequence
  Proteo-36 Oligonucleotide
ccagcgaaaggacggtg

SEQ ID NO: 37 - DNA, 24 bp, Artificial Sequence
  Proteo-37 Oligonucleotide
aatatctttaaaaccggctttcag

SEQ ID NO: 38 - DNA, 18 bp, Artificial Sequence
  Proteo-38 Oligonucleotide
gtgtcctgcatcttggtg

SEQ ID NO: 39 - DNA, 21 bp, Artificial Sequence
  Proteo-39 Oligonucleotide
ggtgttttgcattttagtgtg

SEQ ID NO: 40 - DNA, 17 bp, Artificial Sequence
  Proteo-40 Oligonucleotide
ccagcgagaggatggtg

SEQ ID NO: 41 - DNA, 18 bp, Artificial Sequence
  Proteo-41 Oligonucleotide
cttggcatgcagcatcag

SEQ ID NO: 42 - DNA, 21 bp, Artificial Sequence
  Proteo-42 Oligonucleotide
atgcagaatcagtcgagtaga

SEQ ID NO: 43 - DNA, 23 bp, Artificial Sequence
  Proteo-43 Oligonucleotide
gtgttctgtgctttagagtgaag

SEQ ID NO: 44 - DNA, 18 bp, Artificial Sequence
  Proteo-44 Oligonucleotide
gtgcattacagtagtctg

SEQ ID NO: 45 - DNA, 18 bp, Artificial Sequence
  Proteo-45 Oligonucleotide
gtagccagccttcagcac

SEQ ID NO: 46 - DNA, 18 bp, Artificial Sequence
  Proteo-46 Oligonucleotide
aggctgaggatcgtgtcc

SEQ ID NO: 47 - DNA, 17 bp, Artificial Sequence
  Proteo-47 Oligonucleotide
cagcagcaaggtgcagc

SEQ ID NO: 48 - DNA, 19 bp, Artificial Sequence
  Proteo-48 Oligonucleotide
gccatctccataatggtgt

SEQ ID NO: 49 - DNA, 17 bp, Artificial Sequence
  Proteo-49 Oligonucleotide
ccttgcagccgaggatc

SEQ ID NO: 50 - DNA, 17 bp, Artificial Sequence
  Proteo-50 Oligonucleotide
aggctgaggatggtgtc

SEQ ID NO: 51 - DNA, 18 bp, Artificial Sequence
  Proteo-51 Oligonucleotide
ctcgaggtcttcgacgct

SEQ ID NO: 52 - DNA, 18 bp, Artificial Sequence
  Proteo-52 Oligonucleotide
ctcgcggcaagactcaaa

SEQ ID NO: 53 - DNA, 18 bp, Artificial Sequence
  Proteo-53 Oligonucleotide
ctcgctgcaaggctcaaa

SEQ ID NO: 54 - DNA, 19 bp, Artificial Sequence
  Proteo-54 Oligonucleotide
cgtgtaggatcagccgtgt

SEQ ID NO: 55 - DNA, 20 bp, Artificial Sequence
  Proteo-55 Oligonucleotide
agactaagcacggtgtcttg

SEQ ID NO: 56 - DNA, 19 bp, Artificial Sequence
  Omega-1 Oligonucleotide
tgtctgagccttggcatga

SEQ ID NO: 57 - DNA, 21 bp, Artificial Sequence
  Omega-2 Oligonucleotide
cttcataacgtccttcagttc

SEQ ID NO: 58 - DNA, 19 bp, Artificial Sequence
  Omega-3 Oligonucleotide
ggtccataaccgttgactg

SEQ ID NO: 59 - DNA, 19 bp, Artificial Sequence
  Omega-4 Oligonucleotide
ccattaccgtgttctgtgc

SEQ ID NO: 60 - DNA, 17 bp, Artificial Sequence
  Omega-5 Oligonucleotide
ggtgtcgccatagccga

SEQ ID NO: 61 - DNA, 20 bp, Artificial Sequence
  Omega-6 Oligonucleotide
caaccttcatcacggattcg

SEQ ID NO: 62 - DNA, 17 bp, Artificial Sequence
  Omega-7 Oligonucleotide
cggatgaggtccatcac

SEQ ID NO: 63 - DNA, 19 bp, Artificial Sequence
  Omega-8 Oligonucleotide
gaaccttgtccataaccgt

SEQ ID NO: 64 - DNA, 18 bp, Artificial Sequence
  Omega-9 Oligonucleotide
ctcgcggatcaggtccat

SEQ ID NO: 65 - DNA, 21 bp, Artificial Sequence
  Omega-10 Oligonucleotide
gaccttcatgacatcttccag

SEQ ID NO: 66 - DNA, 18 bp, Artificial Sequence
  Omega-11 Oligonucleotide
acgtccgagagctccaga

SEQ ID NO: 67 - DNA, 19 bp, Artificial Sequence
  Cyano-1 Oligonucleotide
cagtggtttgtgctttaca

SEQ ID NO: 68 - DNA, 22 bp, Artificial Sequence
  Cyano-2 Oligonucleotide
agtgaagaactgttacctgtgc

SEQ ID NO: 69 - DNA, 50 bp, Artificial Sequence
  Cyano-3 Oligonucleotide
gtttgagctttagcgtttaagattagtttgagctttagcgtttaagatta

SEQ ID NO: 70 - DNA, 23 bp, Artificial Sequence
  Cyano-4 Oligonucleotide
aaaccggtcaacattacttcgtg

SEQ ID NO: 71 - DNA, 20 bp, Artificial Sequence
  Cyano-5 Oligonucleotide
gcacctggtttacatacatc

SEQ ID NO: 72 - DNA, 20 bp, Artificial Sequence
  Cyano-6 Oligonucleotide
gcttagtacggttgtttgtg

SEQ ID NO: 73 - DNA, 23 bp, Artificial Sequence
  Cyano-7 Oligonucleotide
tctacacatcggatacctttgta

SEQ ID NO: 74 - DNA, 19 bp, Artificial Sequence
  Cyano-8 Oligonucleotide
ctgcacctttttcagcagc

SEQ ID NO: 75 - DNA, 21 bp, Artificial Sequence
  Cyano-9 Oligonucleotide
tacagtggagcattaagcgag

SEQ ID NO: 76 - DNA, 19 bp, Artificial Sequence
  Cyano-10 Oligonucleotide
cagccaagtgaagaacggt

SEQ ID NO: 77 - DNA, 19 bp, Artificial Sequence
  Cyano-11 Oligonucleotide
cacttaacgccgcgatagc

SEQ ID NO: 78 - DNA, 25 bp, Artificial Sequence
  Cyano-12 Oligonucleotide
attacttcttcgagttcaatatctt

SEQ ID NO: 79 - DNA, 18 bp, Artificial Sequence
  Cyano-13 Oligonucleotide
acgaacgttacggaaacc

SEQ ID NO: 80 - DNA, 22 bp, Artificial Sequence
  Cyano-14 Oligonucleotide
ctaagtggagaatggtagtttg

SEQ ID NO: 81 - DNA, 19 bp, Artificial Sequence
  Cyano-15 Oligonucleotide
ggtgaagaatcgtggtttg

SEQ ID NO: 82 - DNA, 20 bp, Artificial Sequence
  Cyano-16 Oligonucleotide
ggtgtagaattgtggtttgg

SEQ ID NO: 83 - DNA, 19 bp, Artificial Sequence
  Cyano-17 Oligonucleotide
agctaggtgcagaactgtg

SEQ ID NO: 84 - DNA, 18 bp, Artificial Sequence
  Cyano-18 Oligonucleotide
gcagccaaggaaagaacg

SEQ ID NO: 85 - DNA, 18 bp, Artificial Sequence
  Cyano-19 Oligonucleotide
cttcacaccacggaagcc

SEQ ID NO: 86 - DNA, 20 bp, Artificial Sequence
  Cyano-20 Oligonucleotide
cgagcaatacttccgagagt

SEQ ID NO: 87 - DNA, 22 bp, Artificial Sequence
  Cyano-21 Oligonucleotide
acgttgttatagccagtgagta

SEQ ID NO: 88 - DNA, 18 bp, Artificial Sequence
  Cyano-22 Oligonucleotide
gtgcagaatggtggtttg

SEQ ID NO: 89 - DNA, 19 bp, Artificial Sequence
  Cyano-23 Oliognukleotid
cagcagccaattggagaac

SEQ ID NO: 90 - DNA, 18 bp, Artificial Sequence
  Cyano-24 Oligonucleotide
cttttctgctgccaggtg

SEQ ID NO: 91 - DNA, 23 bp, Artificial Sequence
  Cyano-25 Oligonucleotide
agctttacagtgaagaatcaagc

SEQ ID NO: 92 - DNA, 19 bp, Artificial Sequence
  Cyano-26 Oligonucleotide
gagcttcatcaagttccac

SEQ ID NO: 93 - DNA, 17 bp, Artificial Sequence
  Cyano-27 Oligonucleotide
ctggatgccagcgtagc

SEQ ID NO: 94 - DNA, 17 bp, Artificial Sequence
  Frankia-1 Oligonucleotide
atgccccactggccctc

SEQ ID NO: 95 - DNA, 18 bp, Artificial Sequence
  Frankia-2 Oligonucleotide
gccttctcgatgacggtg

SEQ ID NO: 96 - DNA, 18 bp, Artificial Sequence
  ClusterII-1 Oligonucleotide
cccagaatcatgcgggta

SEQ ID NO: 97 - DNA, 23 bp, Artificial Sequence
  ClusterII-2 Oligonucleotide
gttcattccgcctaaaatcatac

SEQ ID NO: 98 - DNA, 21 bp, Artificial Sequence
  ClusterII-3 Oligonucleotide
gttttgatgaccttctcgttg

SEQ ID NO: 99 - DNA, 20 bp, Artificial Sequence
  ClusterII-4 Oligonucleotide
acatccatcagggtttcctg

SEQ ID NO: 100 - DNA, 20 bp, Artificial Sequence
  ClusterII-5 Oligonucleotide
ttgattcatccctcccaaaa

SEQ ID NO: 101 - DNA, 22 bp, Artificial Sequence
  ClusterII-6 Oligonucleotide
tatccatcaatgtttcttgcgg

SEQ ID NO: 102 - DNA, 18 bp, Artificial Sequence
  ClusterII-7 Oligonucleotide
ccatcatggtggtctgca

SEQ ID NO: 103 - DNA, 24 bp, Artificial Sequence
  ClusterII-8 Oligonucleotid
gcattttacctctaagaatcatac

SEQ ID NO: 104 - DNA, 21 bp, Artificial Sequence
  ClusterII-9 Oligonucleotide
gttttccgtggagaatcattc

SEQ ID NO: 105 - DNA, 19 bp, Artificial Sequence
  ClusterII-10 Oligonucleotide
ccgtgcaagattaaccttg

SEQ ID NO: 106 - DNA, 19 bp, Artificial Sequence
  ClusterII-11 Oligonucleotid
gttgtctgcattttaccgc

SEQ ID NO: 107 - DNA, 17 bp, Artificial Sequence
  ClusterII-12 Oligonucleotide
ggtctcctcaggtttgc

SEQ ID NO: 108 - DNA, 17 bp, Artificial Sequence
  ClusterII-13 Oligonucleotide
atcaccgtctgctgcgg

SEQ ID NO: 109 - DNA, 19 bp, Artificial Sequence
  ClusterII-14 Oligonucleotide
ccaggatcagccgatttga

SEQ ID NO: 110 - DNA, 20 bp, Artificial Sequence
  ClusterII-15 Oligonucleotide
caccaaggataagacgagtt

SEQ ID NO: 111 - DNA, 19 bp, Artificial Sequence
  ClusterII-16 Oligonucleotide
cgagcacaagacgagtaca

SEQ ID NO: 112 - DNA, 17 bp, Artificial Sequence
  ClusterIII-1 Oligonucleotide
ccttcttcgcggagcgt

SEQ ID NO: 113 - DNA, 17 bp, Artificial Sequence
  ClusterIII-2 Oligonucleotid
actcggtgcaccggcaa

SEQ ID NO: 114 - DNA, 17 bp, Artificial Sequence
  ClusterIII-3 Oligonucleotide
ccttcctcgcggagtgt

SEQ ID NO: 115 - DNA, 20 bp, Artificial Sequence
  ClusterIII-4 Oligonucleotide
gaagtgattattcctcttcc

SEQ ID NO: 116 - DNA, 19 bp, Artificial Sequence
  ClusterIII-5′Oligonucleotide
cgcctaagagaagacgagt

SEQ ID NO: 117 - DNA, 19 bp, Artificial Sequence
  ClusterIII-6 Oligonucleotide
tcgtcgcgaagggtatcca

SEQ ID NO: 118 - DNA, 17 bp, Artificial Sequence
  ClusterIII-7 Oligonucleotide
ccgcctttacggatatc

SEQ ID NO: 119 - DNA, 16 bp, Artificial Sequence
  ClusterIII-8 Oligonucleotide
ccgcaaggtccacgtc

SEQ ID NO: 120 - DNA, 16 bp, Artificial Sequence
  ClusterIII-9′Oligonucleotide
cttcccggcggatgtc

SEQ ID NO: 121 - DNA, 19 bp, Artificial Sequence
  ClusterIII-10 Oligonucleotide
gcagcgtatccaatacggt

SEQ ID NO: 122 - DNA, 17 bp, Artificial Sequence
  ClusterIII-11 Oligonucleotide
gtcttccccttccgacc

SEQ ID NO: 123 - DNA, 20 bp, Artificial Sequence
  ClusterIII-12 Oligonucleotide
tcttccagatccacgtcttc

SEQ ID NO: 124 - DNA, 19 bp, Artificial Sequence
  ClusterIII-13′Oligonucleotide
cttttctgcgcaagaccac

SEQ ID NO: 125 - DNA, 17 bp, Artificial Sequence
  ClusterIII-14 Oligonucleotide
accctgtccggcggata

SEQ ID NO: 126 - DNA, 19 bp, Artificial Sequence
  ClusterIII-15 Oligonucleotide
gggtatccagaacggactt

SEQ ID NO: 127 - DNA, 19 bp, Artificial Sequence
  ClusterIII-16 Oligonucleotide
acggtccgttgactcaaac

SEQ ID NO: 128 - DNA, 18 bp, Artificial Sequence
  ClusterIII-17 Oligonucleotide
gagccaagccatgcaaca

SEQ ID NO: 129 - DNA, 22 bp, Artificial Sequence
  ClusterIII-18 Oligonucleotide
gtatctaacacactcttttggt

SEQ ID NO: 130 - DNA, 20 bp, Artificial Sequence
  ClusterIII-19 Oligonucleotide
gttctgagtgtatccaacac

SEQ ID NO: 131 - DNA, 18 bp, Artificial Sequence
  ClusterIII-20 Oligonucleotide
ccaaggagaagacgggtg

SEQ ID NO: 132 - DNA, 18 bp, Artificial Sequence
  ClusterIII-21 Oligonucleotide
gtccaagacggtgctttg

SEQ ID NO: 133 - DNA, 19 bp, Artificial Sequence
  ClusterIII-22 Oligonucleotide
tcgagaagattgatggagg

SEQ ID NO: 134 - DNA, 18 bp, Artificial Sequence
  ClusterIII-23 Oligonucleotide
tatccaggacagtgcgct

SEQ ID NO: 135 - DNA, 17 bp, Artificial Sequence
  ClusterIII-24 Oligonucleotide
tccaaaacggtccgctg

SEQ ID NO: 136 - DNA, 17 bp, Artificial Sequence
  ClusterIII-25 Oligonucleotide
ccgaagccctgtttgcg

SEQ ID NO: 137 - DNA, 19 bp, Artificial Sequence
  ClusterIII-26 Oligonucleotide
aaccgggtttacggatatc

SEQ ID NO: 138 - DNA, 17 bp, Artificial Sequence
  ClusterIII-27 Oligonucleotide
aaagccgggcgacacga

SEQ ID NO: 139 - DNA, 22 bp, Artificial Sequence
  ClusterIII-28 Oligonucleotide
cacctaaaagtagacgtgttga

SEQ ID NO: 140 - DNA, 19 bp, Artificial Sequence
  ClusterIII-29 Oligonucleotide
ccaacaacaatcgggttga

SEQ ID NO: 141 - DNA, 21 bp, Artificial Sequence
  ClusterIII-30 Oligonucleotide
cacagcgaacacctttaaaac

SEQ ID NO: 142 - DNA, 19 bp, Artificial Sequence
  ClusterIII-31 Oligonucleotide
cggttttctgctgaagacc

SEQ ID NO: 143 - DNA, 20 bp, Artificial Sequence
  ClusterIII-32 Oligonucleotide
gtcttttgtgctaaaccacc

SEQ ID NO: 144 - DNA, 18 bp, Artificial Sequence
  ClusterIII-33 Oligonucleotide
caccttccgcgagtgtat

SEQ ID NO: 145 - DNA, 20 bp, Artificial Sequence
  ClusterIII-34 Oligonucleotide
agtacggttttctgggctaa

SEQ ID NO: 146 - DNA, 18 bp, Artificial Sequence
  ClusterIII-35 Oligonucleotide
ctcccaataagagacggg

SEQ ID NO: 147 - DNA, 20 bp, Artificial Sequence
  ClusterIII-36 Oligonucleotide
gtatcctaccttcagaaccg

SEQ ID NO: 148 - DNA, 20 bp, Artificial Sequence
  ClusterIII-37 Oligonucleotide
tcaatgtcatcgccttcttc

SEQ ID NO: 149 - DNA, 17 bp, Artificial Sequence
  ClusterIII-38 Oligonucleotide
atgccggaaaagccggg

SEQ ID NO: 150 - DNA, 18 bp, Artificial Sequence
  ClusterIII-39 Oligonucleotide
ccagctctatgtcgtcgc

SEQ ID NO: 151 - DNA, 17 bp, Artificial Sequence
  ClusterIII-40 Oligonucleotide
gtcttctggttcaggcc

SEQ ID NO: 152 - DNA, 19 bp, Artificial Sequence
  ClusterIII-41 Oligonucleotide
cgtatcaagcaccgttttc

SEQ ID NO: 153 - DNA, 24 bp, Artificial Sequence
  ClusterIII-42 Oligonucleotide
caaaatggcatccaattcaatttc

SEQ ID NO: 154 - DNA, 21 bp, Artificial Sequence
  ClusterIII-43 Oligonucleotide
catgatacggtcgagttcaac

SEQ ID NO: 155 - DNA, 21 bp, Artificial Sequence
  ClusterIII-44 Oligonucleotide
ctgagctaatcctcctaaaag

SEQ ID NO: 156 - DNA, 19 bp, Artificial Sequence
  ClusterIII-45 Oligonucleotide
ggtgaagaccaccaagaag

SEQ ID NO: 157 - DNA, 18 bp, Artificial Sequence
  ClusterIII-46 Oligonucleotide
tgctggagtcctcccaac

SEQ ID NO: 158 - DNA, 22 bp, Artificial Sequence
  ClusterIII-47 Oligonucleotide
caagttttcgcacatcatccag

SEQ ID NO: 159 - DNA, 18 bp, Artificial Sequence
  ClusterIII-48 Oligonucleotide
gccgagcttgatgatgtc

SEQ ID NO: 160 - DNA, 22 bp, Artificial Sequence
  ClusterIII-49 Oligonucleotide
cgacttttctaacgtcttcaag

SEQ ID NO: 161 - DNA, 21 bp, Artificial Sequence
  ClusterIII-50 Oligonucleotide
gtacggtcttttggctcaaac

SEQ ID NO: 162 - DNA, 19 bp, Artificial Sequence
  ClusterIII-51 Oligonucleotide
cggttcgcaatgtatccag

SEQ ID NO: 163 - DNA, 18 bp, Artificial Sequence
  ClusterIII-52 Oligonucleotide
caggccgttcagcaaaag

SEQ ID NO: 164 - DNA, 17 bp, Artificial Sequence
  ClusterIII-53 Oligonucleotide
gcctcccagaagcaaac

SEQ ID NO: 165 - DNA, 17 bp, Artificial Sequence
  ClusterIV-1
ccctgatggcgtcaagc

SEQ ID NO: 166 - DNA, 19 bp, Artificial Sequence
  ClusterIV-2 Oligonucleotidee
agtaccgtttcctggtgca

SEQ ID NO: 167 - DNA, 18 bp, Artificial Sequence
  ClusterIV-3
cggttcggttttgccgtc

SEQ ID NO: 168 - DNA, 17 bp, Artificial Sequence
  ClusterIV-4 Oligonucleotide
cctctgagaagggttat

SEQ ID NO: 169 - DNA, 18 bp, Artificial Sequence
  ClusterIV-5 Oligonucleotide
gatgcctttgtagccggt

SEQ ID NO: 170 - DNA, 18 bp, Artificial Sequence
  ClusterIV-6 Oligonucleotide
ctgcgtgttgtcccgtat

SEQ ID NO: 171 - DNA, 18 bp, Artificial Sequence
  ClusterIV-7 Oligonucleotide
ttctgcaaggatccgcgt

SEQ ID NO: 172 - DNA, 17 bp, Artificial Sequence
  ClusterIV-8 Oligonucleotide
caccgcggtgatgatcc

SEQ ID NO: 173 - DNA, 16 bp, Artificial Sequence
  ClusterIV-9 Oligonucleotide
tcatgggcattgaccg

SEQ ID NO: 174 - DNA, 17 bp, Artificial Sequence
  ClusterIV-10 Oligonucleotide
ccgtccctcatgctgtc

SEQ ID NO: 175 - DNA, 17 bp, Artificial Sequence
  ClusterIV-11 Oligonucleotide
ttgccgcctgtcaggtt

SEQ ID NO: 176 - DNA, 18 bp, Artificial Sequence
  ClusterIV-12 Oligonucleotide
cctccgctttcaacacat

SEQ ID NO: 177 - DNA, 18 bp, Artificial Sequence
  ClusterIV-13 Oligonucleotide
gcctctcaccatagagtg

SEQ ID NO: 178 - DNA, 24 bp, Artificial Sequence
  ClusterIV-14 Oligonucleotide
caatatcaaggatagtaggcaatc

SEQ ID NO: 179 - DNA, 20 bp, Artificial Sequence
  ClusterIV-15 Oligonucleotide
ctttccgtgcattaatgtcc

SEQ ID NO: 180 - DNA, 18 bp, Artificial Sequence
  ClusterIV-16 Oligonucleotide
aatcctacgtccagcgag

SEQ ID NO: 181 - DNA, 21 bp, Artificial Sequence
  ClusterIV-17 Oligonucleotide
ctgtattttgtgtccgcatac

SEQ ID NO: 182 - DNA, 18 bp, Artificial Sequence
  ClusterIV-18 Oligonucleotide
accgtggggatcttcctc

SEQ ID NO: 183 - DNA, 18 bp, Artificial Sequence
  Cluster-IV-19 Oligonucleotide
actgtgggaatgtcagcc

SEQ ID NO: 184 - DNA, 20 bp, Artificial Sequence
  ClusterIV-20 Oligonucleotide
taatatcctgacccttttgc

SEQ ID NO: 185 - DNA, 17 bp, Artificial Sequence
  ClusterIV-21 Oligonucleotide
aggtaatccagtaccgt

SEQ ID NO: 186 - DNA, 18 bp, Artificial Sequence
  ClusterIV-22 Oligonucleotide
ccgtgcaacaacagtttc

SEQ ID NO: 187 - DNA, 18 bp, Artificial Sequence
  ClusterIV-23 Oligonucleotide
gccaaggctttccagcat

SEQ ID NO: 188 - DNA, 19 bp, Artificial Sequence
  ClusterIV-24 Oligonucleotide
tgacctcctggacgttatc

SEQ ID NO: 189 - DNA, 19 bp, Artificial Sequence
  ClusterIV-25 Oligonucleotide
ccgccccttaaatttgaag

SEQ ID NO: 190 - DNA, 16 bp, Artificial Sequence
  Oligonucleotide A289
gggccttgttttcgcg

SEQ ID NO: 191 - DNA, 23 bp, Artificial Sequence
  Oligonucleotide A1(6A)
aaaaaatgcgacccgaaggctga

SEQ ID NO: 192 - DNA, 23 bp, Artificial Sequence
  Oligonucleotide A4(6A)
aaaaaagagtcagccttcgggtc

SEQ ID NO: 193 - DNA, 23 bp, Artificial Sequence
  Oligonucleotide A8(6A)
aaaaaaggtggagtcagccttcg

SEQ ID NO: 194 - DNA, 23 bp, Artificial Sequence
  Oligonucleotide A14(6A)
aaaaaacagacgggtggagtcag

SEQ ID NO: 195 - DNA, 23 bp, Artificial Sequence
  Oligonucleotide A20(6A)
aaaaaagaggatcagacgggtgg

SEQ ID NO: 196 - DNA, 22 bp, Artificial Sequence
  Oligonucleotide A64(6A)
aaaaaacttcagcggccaggtg

SEQ ID NO: 197 - DNA, 22 bp, Artificial Sequence
  Oligonucleotide A114(6A)
aaaaaagaagccgaccgacagg

SEQ ID NO: 198 - DNA, 23 bp, Artificial Sequence
  Oligonucleotide A188(6A)
aaaaaaggcggtgataacgccac

SEQ ID NO: 199 - DNA, 24 bp, Artificial Sequence
  Oligonucleotide A235(6A)
aaaaaagaagtcgagttcgtcgtc

SEQ ID NO: 200 - DNA, 22 bp, Artificial Sequence
  Oligonucleotide A 307(6A)
aaaaaagggccttgttttcgcg

SEQ ID NO: 201 - DNA, 23 bp, Artificial Sequence
  Oligonucleotide S20(6A)
aaaaaaccacccgtctgatcctc

SEQ ID NO: 202 - DNA, 22 bp, Artificial Sequence
  Oligonucleotide S114(6A)
aaaaaacctgtcggtcggcttc

SEQ ID NO: 203 - DNA, 22 bp, Artificial Sequence
  Oligonucleotide S307(6A)
aaaaaacgcgaaaacaaggccc

SEQ ID NO: 204 - DNA, 29 bp, Artificial Sequence
  Oligonucleotide A20(12A)
aaaaaaaaaaaagaggatcagacgggtgg

SEQ ID NO: 205 - DNA, 28 bp, Artificial Sequence
  Oligonucleotide A307(12A)
aaaaaaaaaaaagggccttgttttcgcg

SEQ ID NO: 206 - DNA, 23 bp, Artificial Sequence
  Oligonucleotide A20(6A)3′
gaggatcagacgggtggaaaaaa

SEQ ID NO: 207 - DNA, 22 bp, Artificial Sequence
  Oligonucleotide A307(6A)3′
gggccttgttttcgcgaaaaaa

SEQ ID NO: 208 - DNA, 17 bp, Artificial Sequence
  nifH PCR forward primer
tgygayccnaargcnga

SEQ ID NO: 209 - DNA, 17 bp, Artificial Sequence
  nifH reverse primer
adngccatcatytcncc

SEQ ID NO: 210 - DNA, 17 bp, Artificial Sequence
  nifH PCR forward primer
tgcgacccgaaggctga

SEQ ID NO: 211 - DNA, 16 bp, Artificial Sequence
  nifH PCR forward primer
cctgtcggtcggcttc

SEQ ID NO: 212 - DNA, 16 bp, Artificial Sequence
  nifH reverse primer
gggccttgttttcgcg

SEQ ID NO: 213 - DNA, 50 bp, Artificial Sequence
  Oligonucleotide A19-68
aggtgcatcacggtggtctgggccttgctgtggaggatcagacgggtgga

SEQ ID NO: 214 - DNA, 50 bp, Artificial Sequence
  Oligonucleotide A289-338
acgatgtagatttcctgggccttgttttcgcgaatcggcatcgcgaagcc

SEQ ID NO: 215 - DNA, 50 bp, Artificial Sequence
  Oligonucleotide S19-68
tccacccgtctgatcctccacagcaaggcccagaccaccgtgatgcacct

SEQ ID NO: 216 - DNA, 50 bp, Artificial Sequence
  Oligonucleotide S289-338
ggcttcgcgatgccgattcgcgaaaacaaggcccaggaaatctacatcgt

(19) United States
(12) Patent Application Publication
(10) Pub. No.: US 2008/0096196 A1
(43) Pub. Date: Apr. 24, 2008

(54) Method for Analyzing Samples by Means of Hybridization

(76) Inventors: Lei Zhang, Bremen (DE); Barbara Reinhold-Hurek, Bremen (DE); Thomas Hurek, Bremen (DE)

Correspondence Address: RANKIN, HILL & CLARK LLP, 925 EUCLID AVENUE, SUITE 700, CLEVELAND, OH, 44115-1405, US

(21) Appl. No.: 11/572,854

(22) PCT Filed: Jul. 27, 2005
(86) PCT No.: PCT/EP05/08150

§ 371 Date: Sep. 17, 2007

(30) Foreign Application Priority Data
Jul. 30, 2004 (DE) 10 2004 037 081.8
Apr. 22, 2005 (DE) 10 2005 018 871.0

(51) Int. Cl. C12Q 1/68; C07H 21/04; G01N 33/566

(52) U.S. Cl. 435/6; 436/501; 536/23.1

(57) ABSTRACT

The invention relates to a method of analysing samples by means of a ligand binding, in which duplexes or complexes are created and analysed.

The invention is distinguished by the fact that the target molecules have a detectable marking in proximity to a target sequence and/or a detectable marking is incorporated in the target sequence. By this means, significantly higher signal intensities are obtained than with conventional methods.

The present invention relates to a method of analysing samples by means of a ligand binding method, target molecules which may be used in such methods, and kits for the provision of such target molecules and/or implementation of the aforementioned method.

### BACKGROUND OF THE INVENTION

Nucleic acids are normally present in the form of double stranded molecules, also described as heteroduplexes and comprised of two nucleic acid molecules. If duplexes are subjected to a rise in temperature, they dissociate into two single-stranded nucleic acid molecules. With a lowering of temperature, these may re-associate to form a heteroduplex. The direct reaction to duplexes is described as hybridization or re-hybridization. Duplexes are also described as hybrids.

Nucleic acids are polymers of the nucleotides adenine, cytosine, guanine, thymin, uracil, inosin (a, c, g, t, u, i), synthetic or modified nucleotides, arranged next to one another in the polymer like pearls in a chain of pearls. The sequence of the nucleotides in a nucleic acid molecule is specific for that particular nucleic acid molecule. Double strands are formed through hydrogen bonds, which may develop for example between individual nucleotides a and t, also c and g, if on a single strand there are sufficient nucleotides in succession meeting a particular counter-nucleotide on another single strand—and there in the corresponding sequence. In nature, nucleic acids in the form of such complementary single strands occur as double strands.

One is frequently faced with the problem that the existence of a specific nucleic acid in a reaction mixture or a biological sample (hereafter combined under the term “reaction mixture”) needs to be detected. This is generally done by using catcher molecules, which have single-stranded nucleic acids which hybridise to form duplexes with a single-stranded or partly single-stranded nucleic acid molecule, hereafter known as the target molecule. The detection of the duplex permits a statement on the presence of the target molecule. Since in nucleic acids certain sequences can and do occur several times, the catcher molecule is so chosen that it reacts preferably with a target sequence which represents a section of the total sequence of the nucleic acid to be detected, and is as specific as possible for it. In so doing, an attempt is generally made to ensure the most rigorous possible proof of the catcher-target sequence hybrids or duplexes, and the greatest possible specificity or uniqueness of the target sequence for the nucleic acid concerned.

The detection of nucleic acids using catcher-target sequence hybrids has been known for many years. It has developed from the so-called Southern Blot through many variants to oligonucleotide chips or DNA micro-arrays, in which on a few cm² thousands of oligonucleotide sequences are provided as physically separated spots at a fixed phase, and function as catcher molecules. Many of these spots allow, parallel in time, a qualitative and to a limited extent quantitative statement on the presence or absence of certain sequences in a reaction mixture. In addition, there is a multiplicity of variations of the catcher-target sequence hybrid detection principle, which are generally known to the experts in this field.

To make possible the detection of a catcher-target sequence hybrid, an adequate number of detectable hybrids are needed.

Frequently one is confronted with small sample quantities, which must be amplified by a PCR-reaction over several cycles. Each cycle of a PCR-reaction is equivalent to a copying process, in which the number of copies increases exponentially with each step. Each step costs time and material and, the longer the copying process is continued, the more likely the copies are to be defective, The fewer cycles are needed to produce a detectable sample quantity, the more advantageous and reliable will be the hybridization experiment conducted with a sample prepared in this way.

In the quantification of hybrids there are considerable imponderables. The maximum number of hybrids in a probe spot or spot of a micro-array may not exceed the number of catcher molecules in the spot. This is generally known. It is however difficult to make reliable statements as to how many catcher molecules of a spot will form catcher-target sequence molecules in an experiment. Quite a few of the factors which affect the efficiency of hybridization are already known (Southern et al., 1999), such as e.g. the distance between the sequence of the catcher oligonucleotide complementary to the target molecule and the glass surface. A worsening of efficiency when the duplex leads to an excessive overhang of the target molecule in the direction of the glass surface has also been described as a steric effect (Peplies et al. 2003). The intensity of the detection signals used to detect the presence of hybrids does indeed seem to be roughly proportional to the number of hybrids present in a probe spot, but the proportionality factor is apparently not identical from one probe spot to another, when different hybrids are present in different probe spots.

EP 0721016 A2 describes a method for the discrimination of perfectly complementary hybrids from those which differ in one or more bases. Through enzymatic decomposition of single-stranded polynucleotides after hybridization reaction, discrimination is made between perfectly complementary hybrids and those with incorrect pairings. Only perfectly complementary and double stranded hybrids are still present after decomposition and may be detected with the aid of their fluorescent marking. Marked RNA target molecules may be produced by means of standard PVR or in vitro transcription, with approximately 10% of the uracil of the target sequence fluorescein being marked at random. A second method relates to the detection of perfect hybrids through ligation of marked oligonucleotides, after hybridization with the target molecule has taken place. After ligation, the target molecule is once again separated from the catcher molecule and washed. Detection of the remaining single strands then takes place.

A similar method is described in WO 98/23776. Here, target molecules should be hybridized by a varying number of repetitions with catcher molecules of known length. After digestion by exonuclease, only perfect hybrids remain, so that the number of repetitions may be determined. Once again only the perfectly complementary double strand may be detected with the aid of its marking. The target molecule is marked. This marking may be inserted during the PCR with marked primer or marked nucleotides or by other methods. The marking is applied at any desired position and preferably at the 5′ or 3′ end.

WO 98/53103 describes DNA arrays with various polynucleotides within individual spots, and kits with such DNA arrays, together with their production and use. Each of the spots on a solid substrate belongs to a specific gene type (e.g. heavily regulated gene, or gene associated with specific stages of sickness). The target molecules to be hybridized may be produced by all known methods, with the use of primers specific for the gene to be analysed being proposed. The target molecules are marked, and the marking may be located in the gene-specific primer or in the dNTPs. Here the length of the catcher molecules in the array typically amounts to 120-800 bases, which represents only a portion of the overall length of the cDNA (target molecules) to be analysed.

WO 01/23600 A2 on the other hand is concerned with a method for the quantification of relative specificities of the hybridization reaction with the aid of dissociation curves. At least a portion of the detectable marked target molecules is in this case at least partly complementary to the samples. The dissociation curve of a perfectly complementary sequence may be used e.g. as reference. The difference in the integral of the dissociation curve to be analysed to the reference curve is a function of specificity and is used as the measure. Detectable marked target molecules are here hybridized to the samples and subsequently washed in stages, with the dissociation curve resulting from the signal intensity. The method may be used with all types of marked polynucleotides.

The publication Peplies et al., Applied and Environmental Microbiology (69), 3, pp. 1397-1407, 2003 describes a study which systematically investigates the applicability of arrays to questions of microbiological ecology. In order to decide which factors influence the specific recognition of sections of the 16SrRNA gene and lead to false positive and false negative results, the authors use twenty different catcher molecules of 15-20 bases in length, in which it is known that the 16SrRNA gene of different species differs. The target molecules are produced by amplification the 16SrRNA gene of six typical bacterial strains with the aid of marked gene-specific primers. The hybridization between the target molecules and the corresponding catcher molecules then takes place in different areas of the target molecules marked at the 5′ end. The marking therefore has sharply varying distances from the catcher molecule in the different spots on the array. It should be located outside the hybridization areas (5′ end base 8: see Materials and Methods—preparation of fluorescently labelled target single-stranded DNA: “5′-indocarbocyanine-labelled forward primer 8f” and Table 1: “16SrRNA binding site and length”).

U.S. Pat. No. 5,871,928 A describes methods of sequencing, fingerprinting and plotting biological macro-molecules. Since the position and sequence of a probe on an array is known, sequencing of marked target molecules may be undertaken. In this, e.g. overlapping probe nucleotides of five bases in length are used. The marked target molecule binds to different probes and the sequence of the target molecule may be determined through overlapping of the probe sequence. The marking of the signal molecule is achieved by standard methods. The marked signal molecule may be fragmented to enhance the signal. The signal enhancement effect results from a higher concentration of marked hybridized fragments per probe sequence. A relatively long target molecule is also detectable with a relatively small number of markings per unit length since, on account of the length, many markings are available.

U.S. Pat. No. 6,027,889 A relates to a method for the detection of nucleic acid sequences through the coupling of ligase with PCR-reactions. Two target sequences, which bind next to one another at a sequence to be analysed and also contain an overhang, are ligated. After ligation, the overhangs are used in a PCR-reaction for the hybridization of a marked ZIP code primer. The amplified DNA may be analysed by various methods (gel filtration, arrays, etc.). The marking is introduced by means of PCR, with one primer carrying the marking and the other primer being linked to the hybridisable sequence, so that hybridization and marking are spatially separate from one another. Ligation reaction and PCR-reaction may be combined in different ways for various applications.

The problem of the present invention is therefore to provide a method for the analysis of samples by means of hybridization, in which even very small sample quantities may be detected more reliably and clearly than before, and in which the detected signals may be better correlated with one another than is the case with known methods.

The problem is solved by a method of analysing samples by means of a ligand binding method in which, through the binding of target sequences of target molecules to catcher sequences of probes, duplexes or complexes are generated and/or duplexes or complexes thus generated are analysed, wherein the target sequence is a partial sequence of the target molecule and the duplexes or complexes have at least one detectable marking or an accumulation of markings in proximity to and/or within the target sequence.

Surprisingly it has been found that the use of catcher sequences complementary to target sequences of the target molecules which have a detectable marking or an accumulation of markings in proximity to and/or within the target sequence lead, in methods according to the invention, to signal intensities considerably higher than those obtained in comparable methods using catcher sequences not selected according to the invention, so that the marking is not in proximity to the target sequence.

Preferably the detectable marking or the accumulation of markings is located only in proximity to and/or within the target sequence.

Target molecules may be marked with a single marking. In principle, though, it is also customary to provide target molecules with several markings. Here the accumulation of markings is to be provided in proximity to and/or within the target sequence.

For the purposes of the present invention, an accumulation of markings is understood to mean preferably the maximum accumulation of markings on the target molecule, to be found within an area which is not longer than the target sequence and permits a specific duplex formation. In principle it is possible for the target molecule to be provided with further markings or accumulations of markings which however do not always lie in areas which are specific for the target molecule and are therefore not suitable as target sequence.

Due to the fact that the target sequence is limited to areas specific to the target molecule, it is often not freely variable, for which reason according to the invention at least one marking is provided in proximity to or within an area specific to the target molecule, and the catcher sequence is determined complementary to this area, and then represents the target sequence.

In a method according to the invention, several samples are analysed simultaneously by means of ligand binding, wherein all target molecules have either at least one detectable marking or an accumulation of markings in proximity to the relevant target sequence.

In another method according to the invention, all target molecules have the same number of markings.

In methods according to the invention, at least ten samples, preferably at least one hundred samples or at least a thousand samples are analysed simultaneously.

In methods according to the invention, the marking may be a fluorescent marking.

In methods according to the invention, the fluorescent marking is obtained by means of one or more of the following marking agents: Cy3, Cy5, fluorescein, Texas red, Alexa fluor dyes and other fluorescent dyes.

The problem is also solved by a method for the production of target molecules which have at least one marking only in proximity to the relevant target sequence or within the relevant target sequence, wherein the target sequences are partial sequences of the relevant target molecule.

Used in the invention are target molecules which comprise and/or have target sequences and a marking in proximity to or within the target sequence concerned.

These target molecules may be used in the methods according to the invention described above and lead to catcher-target sequence hybrids with a greater signal intensity than hybrids produced with the assistance of target molecules which have target sequences and a marking for the relevant target sequence which is not in proximity to the latter.

The marking of the target molecules may be effected by means of enzymatic end marking, reverse transcription, indirect marking and/or “sandwich” methods.

The problem is also solved by a method for the generation of target molecules involving a PCR method in which at least one primer provided with a marking agent is used.

In the method according to the invention, at least one further primer provided with an identical or another marking agent may be used. By this means, different types of signal may be provided, in order to verify results in methods according to the invention, but also to match to one another the intensity of different spots containing varying numbers of hybrids, so that the intensities lies in the same order of magnitude. The spot containing more hybrids may have for example the marking agent with the lower signal intensity. Since the signals may be distinguished from one another on the basis of different marking agents, and the factor by which the signal intensities differ from one another is known, such a method makes possible quantification and a better comparison of the intensities in both spots. This is of particular benefit when the use of a marking agent with a greater signal intensity would lead to a saturation value in the detection unit, for example a photo-multiplier. In such a case, quantification would be impossible or at least more difficult due to factors relating to the apparatus used.

If in a PCR-method, use is made of primers which are marked and dNTPs which have no markings, then the PCR-product is marked at the end formed by the primers, and unmarked at other points. If the primers are selected so that the target sequence of the PCR-product is in proximity to the primer, then the PCR-product represents a target molecule which has a marking according to the invention only in proximity to the target sequence. This is an extremely simple and uncomplicated method of obtaining target molecules according to the invention. By using defined primers of a specific length, it is possible to make a more accurate statement as to how much marking agent has been incorporated into each target molecule than if a mixture of marked and unmarked dNTPs is used to produce a marked PCR-product; for if individual dNTPs are marked it is not always clear how many marked and how many unmarked dNTPs are incorporated in a PCR-product, or else the PCR-products differ in respect of the marked nucleotides incorporated in them. This is also dependent on the particular sequence amplified. Usually one of the four bases to be incorporated is marked. If this appears less often in an amplification product, then the amplification product contains less marking agent than other amplification products. Because of this fact it is considerably more difficult to make quantitative statements on the basis of a micro-array experiment. With suitable choice of primer it is possible to ensure that each primer used in an experiment has roughly the same amount of marking agent. These amounts no longer vary on the basis of a target sequence to be amplified, but depend solely on the composition and form of the particular primer used. Apart from the enhanced signal intensities which improve considerably the sensitivity of micro-array experiments, methods according to the invention also represent, for the reasons given above, significantly more accurate methods than methods known to date from the prior art.

In a method according to the invention for the production of target molecules according to the invention, target molecules are generated by means of a PCR method in which at least one type of dNTP provided with a marking agent is used, and in which the marked dNTPs are incorporated in the target molecule in direct proximity to the target sequence and/or in the target sequence itself.

In contrast to the prior art, such a method according to the invention ensures that the marking agent is actually present in direct proximity to the target sequence. In this connection, the target sequence represents only a section or a partial sequence of the target molecule. The length of the target molecule is generally considerably more than that of the target sequence, and in particular 2, 3, 4 or 5 times the length of the target sequence. In target molecules of such length, a random distribution of markings leads to significantly lower signal intensities than is the case when the markings are positioned according to the invention. This is shown very impressively by the examples explained below. In this context, direct proximity to the target sequence means that marking agent is bound to nucleotides incorporated in the target sequence or to nucleotides incorporated adjacent to the target sequence. For the purposes of the invention, direct proximity to the target sequence means that the marking or the marking agent is no further than 100 or 60, and preferably no further than 0-20 bases distant from the target sequence. Significant positive effects (factors 2-146) may still be detected at a distance of 100 bases. Preferably the marking agent is located only in direct proximity to the target sequence.

Other methods according to the invention comprise the fluorescent marking of nucleic acids (DNA or RNA) by means of other methods, e.g. by enzymatic end marking (e.g. using terminal transferase, polynucleotide kinase, poly(a)-polymerase or other enzymes or by reverse transcription with fluorescent-marked primers, or by indirect marking methods.

A further method according to the invention for the production of target molecules according to the invention is one in which the target molecules are generated by means of a PCR method and in which at least one type of dNTP provided with a marking agent is used, and in which the marked dNTPs or an accumulation thereof are incorporated in direct proximity to the target sequence and/or in the target sequence itself, and in which one or more primers is provided with the same or other marking agents .

Other methods according to the invention for the production of target molecules according to the invention are methods in which marking agents are incorporated in direct proximity to the target sequence and/or in the target sequence itself. Here many methods are possible, e.g. incorporation of marked dNTPs in cDNA through reverse transcription, or post-labelling protocols which make use of the incorporation of aminoallyl-nNTP in cDNA and then a chemical coupling with fluorescent dyes, or direct non-enzymatic marking of the RNA with fluorescent dyes.

Such a method leads to target molecules according to the invention which are still easily detected even with hybrids formed at very low concentrations since, due to the incorporation of marking agent in the target sequence, a comparably large amount of marking agent may be incorporated. At least with target molecules present in normal concentrations, the result in one spot may also be verified with the aid of the additional marking agent in the primer used.

The problem is also solved by target molecules, for use in a method according to the invention, which have a marking or an accumulation of markings in proximity to or within the relevant target sequence, with the target sequence forming a section or a partial sequence of the target molecule.

Target molecules according to the invention provided by methods according to the invention are preferred.

The problem is finally solved by a method according to the invention in which target molecules according to the invention are used, which also have been or may be produced by a method according to the invention for the production of such molecules.

A set of catcher molecules according to the invention for the implementation of methods according to the invention comprises:
a predetermined number of different catcher molecules comprising in each case different catcher sequences, each designed to from ligand bonds with target sequences of target molecules in such a way that they are complementary to the relevant sections of the target sequences to be found in proximity to a marking or an accumulation of markings.

A set of catcher molecules according to the invention has at least 10, 30, 50, 100, 1000 or 5000 different catcher sequences. Preferably all catcher molecules of the set of catcher molecules are designed so that they are complementary to the relevant sections of the target sequences which are in proximity to a marking or an accumulation of markings.

In a set according to the invention, the catcher sequences are constituents of the probes of a DNA array.

According to a method according to the invention, the catcher molecules are so determined and calculated that they are complementary to the relevant sections of the target sequences which are in proximity to a marking or an accumulation of markings.

Here a target sequence is defined as an area of the target molecule which is specific to the target molecule.

In one embodiment, the method according to the invention may be used for the identification of N₂-fixing organisms by means of sequence analysis. N₂-fixing organisms contain nitrogenases for the reduction of atmospheric nitrogen to ammonium, the coded genes of which are suitable for phylogenetic sequence analysis. For this purpose it is now possible to use diagnostic arrays containing catcher molecules according to the invention, which bind to regions of the nitrogenase gene which are specific for certain nitrogenase groups. Here the specificity of a catcher oligonucleotide may cover larger phylogenetic or other groups, and smaller groups such as e.g. species, types or individual strains.

Especially suitable for such a phylogenetic sequence analysis is the enzyme nitrogenase, in particular the subunit coded by the gene nifH (Hurek et al., 1997; Hurek et al., 2002). Through sequence analyses of nifH—including genes for alternative nitrogenase anfH and vnfH—it is possible to detect and identify nitrogen-fixing procaryotes without cultivation in environmental samples in DNA preparations. The subunit nifH, for example, is highly suitable for simultaneous amplification of all phylogenetically different nifH variants by means of PCR (Tan, Hurek, Reinhold-Hurek 2003) owing to the relatively high degree of conservation and the very well matured primer systems. Even the most active N₂-fixing bacteria may be detected if the analyses are made on the mRNA level (Hurek et al., 2002). The analysis of the diversity of nitrogenase genes may be significantly reduced, in comparison with sequencing, if diagnostic micro-arrays are used. Thus, the invention also covers the use of a diagnostic micro-array, with the catcher molecules binding to regions of nifH/anfH/vnfH genes which are specific for certain nitrogenase gene groups.

Suitable catcher molecules for this sequence analysis according to the invention may be obtained by firstly arranging (alignment) for known genes the nifH, anfH or vnfH gene areas which are amplified by the PCR primers used. Here the protein sequence is taken into account, i.e. the bases lie in a position which in each case codes the amino acid concerned. With the aid of this alignment and after creating a phylogenetic pedigree, it is possible from this to select as target sequences the sequences which are identical in a target gene group and may be delimited from all other known sequences, with the delimitation being effected through the presence of mismatch positions.

Preferably the target sequences are so selected that there are at least two mismatch positions, one of which may lie centrally in the target sequence which delimits the target group of nitrogenase genes from other nitrogenase genes, while at least one other mismatch position is to be found in a non-target sequence.

It is also advantageous for the target sequences to have a Tm value which is as similar as possible to the other target sequences, preferably 60-70° C., with 62-69° C. being especially preferred.

The sequences with reverse complementarity to the above are then selected as catcher sequences for the catcher molecules.

To obtain specific detection with high phylogenetic resolution, in particular at the species or type level, catcher molecules of up to 35 mer, preferably up to 30 mer with 17-25 mer being especially preferred, may be used. The short catcher molecules increase the rigorousness of the hybridization, making small sequence variations such as occur among different types and/or species detectable.

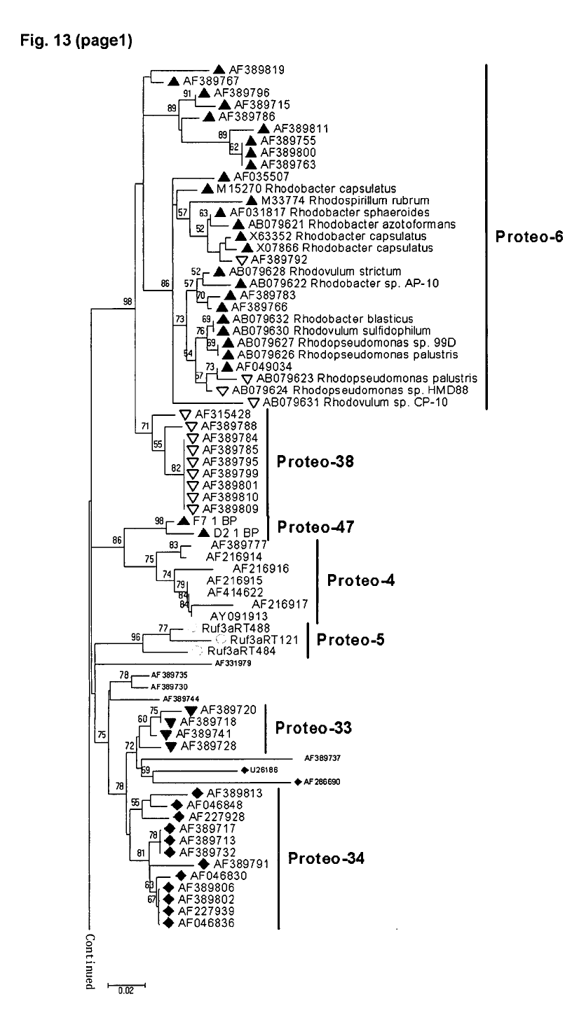

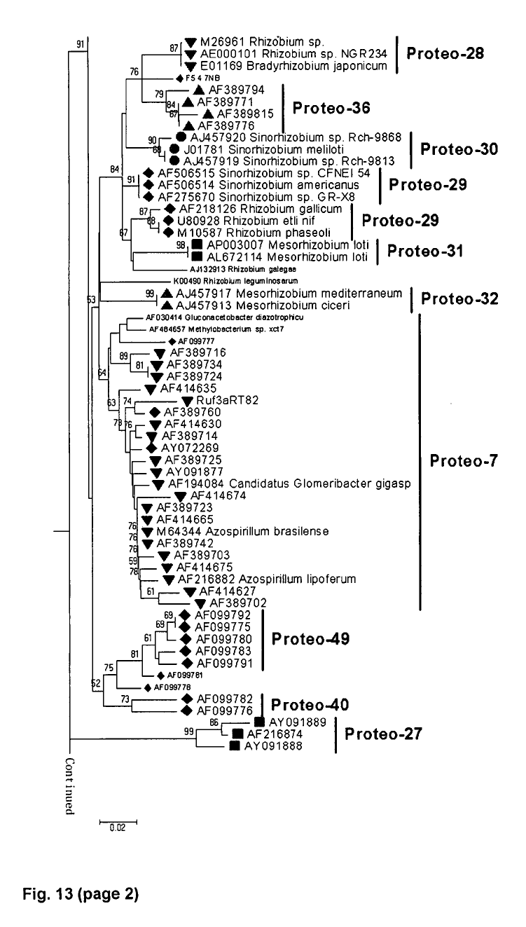

Examples of suitable catcher molecules (SEQ ID NO: 1-189) for the method of sequence analysis referred to above are listed in Table 1. The specificities of the catcher molecules (SEQ ID NO: 1-189) of Table 1 are to be found in FIGS. 13 to 18. The catcher molecules of SEQ ID NO: 1-189 have been selected so that they bind as close as possible to the marked end of the target molecule and at the same time have the desired specificity (FIGS. 13 to 18). Besides the oligonucleotides (SEQ ID NO: 1-189) listed in Table 1, according to the invention oligonucleotides are also suitable as catcher molecules if they differ in their length by one or more nucleotides from the oligonucleotides of SEQ ID NO: 1-189, or if their nucleotide sequence differs at one or more positions from the corresponding oligonucleotides of SEQ ID NO: 1-189, so long as they have the same specificity as the corresponding oligonucleotides of SEQ ID NO: 1-189, i.e. they bind at the same respective nitrogenase gene regions listed in FIGS. 13 to 18 as the corresponding oligonucleotide of Table 1.

To produce the arrays for sequence analysis according to the invention, nitrogenase gene-specific catcher molecules are immobilised on a matrix (slide).

### PRECISE DESCRIPTION OF THE INVENTION

The invention is explained in detail below with the aid of the figures and a number of embodiments. The figures show in:

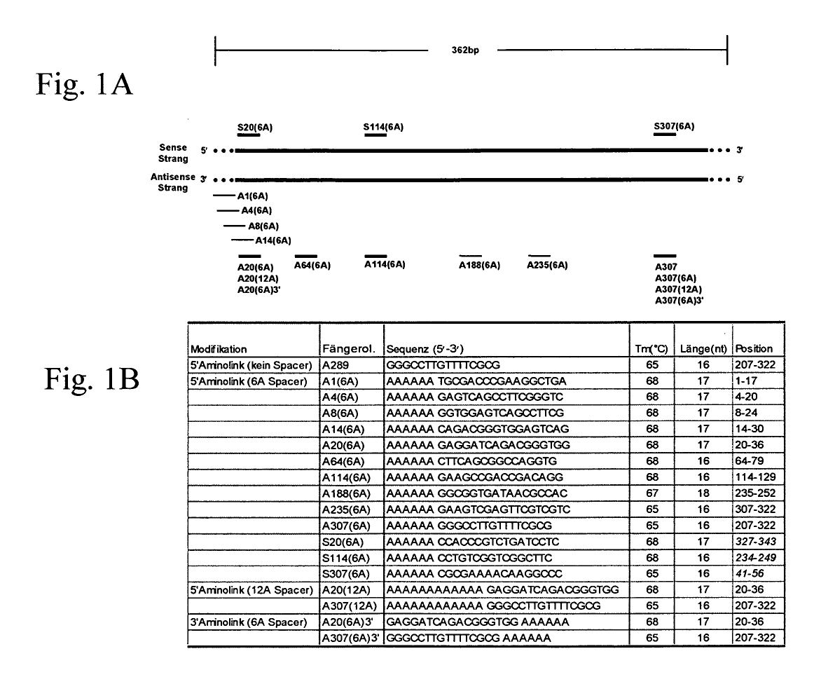

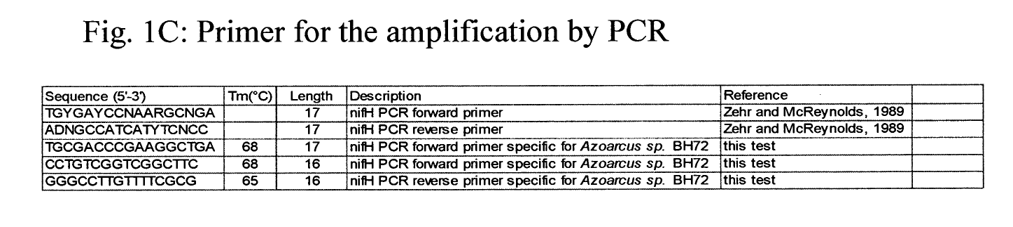

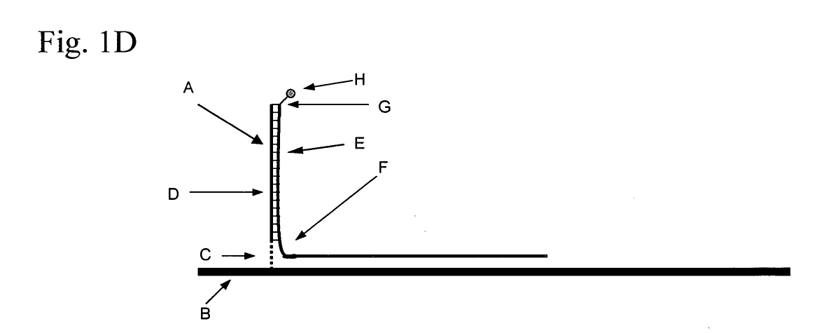

FIG. 1a a 362 bp long target molecule according to one of the embodiments, in schematic form

FIG. 1b a table listing oligonucleotides as used in the embodiments

FIG. 1c a table listing oligonucleotides as used in the embodiments as primers for amplification by means of PCR

FIG. 1d the model system in schematic form

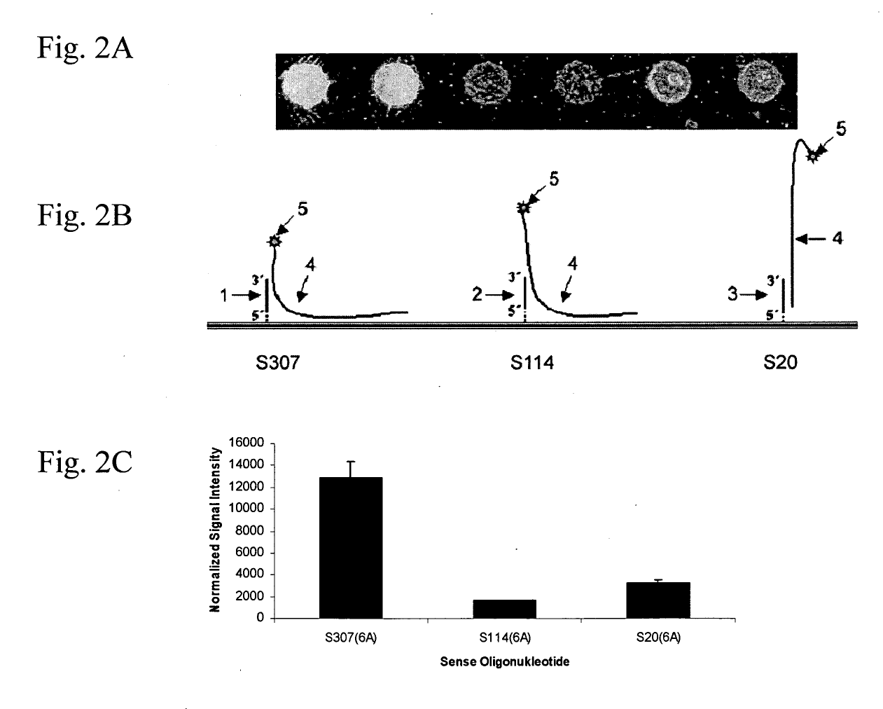

FIG. 2a fluorescence signals of the hybridization of a Cy5-marked antisense strand with sense oligonucleotides

FIG. 2b a schematic representation of the hybrid molecules

FIG. 2c a graphical representation of determined signal intensities

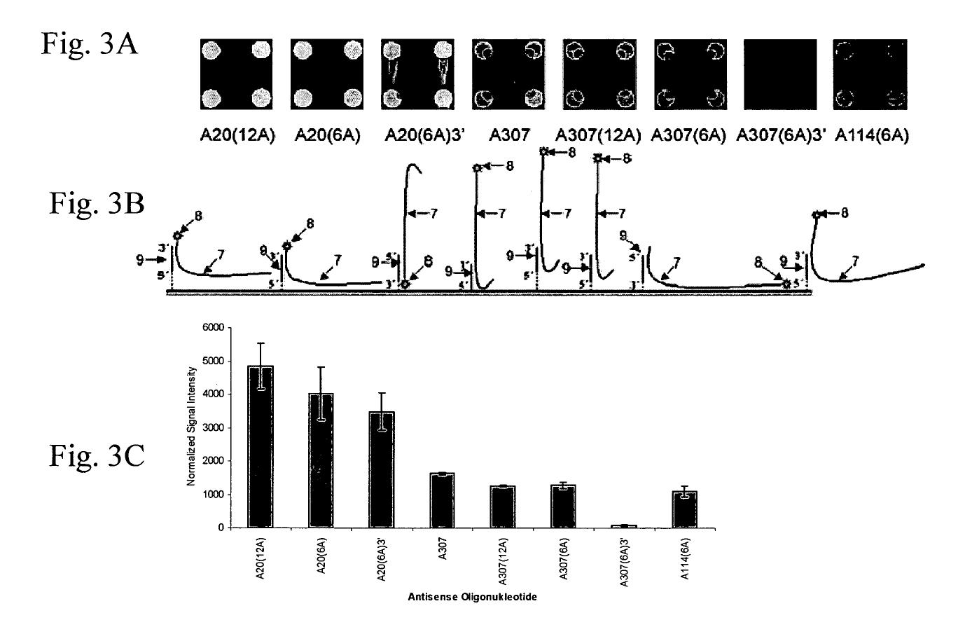

FIG. 3a fluorescence signals of the hybridization of a Cy5-marked sense strand with antisense oligonucleotides

FIG. 3b a schematic representation of the hybrid molecules belonging to FIG. 3a

FIG. 3c a graphical representation of the signal intensities of the fluorescence signals of FIG. 3a

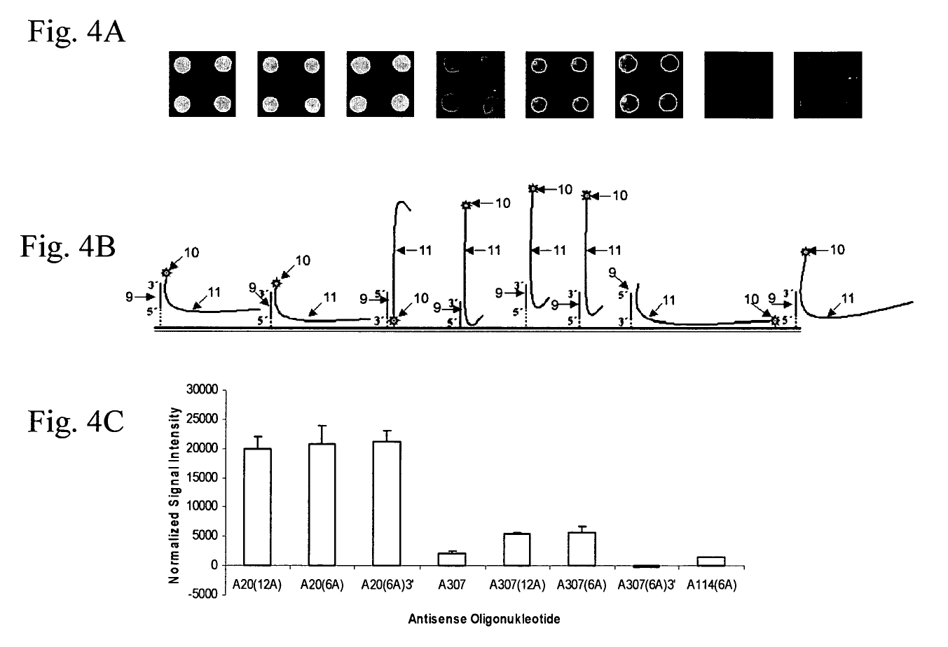

FIG. 4a fluorescence signals of a hybridization of a Cy3-marked sense strand with antisense nucleotides

FIG. 4b a schematic representation of the hybrid molecules of FIG. 4a

FIG. 4c a graphical representation of the signal intensities of the fluorescence signals of FIG. 4a

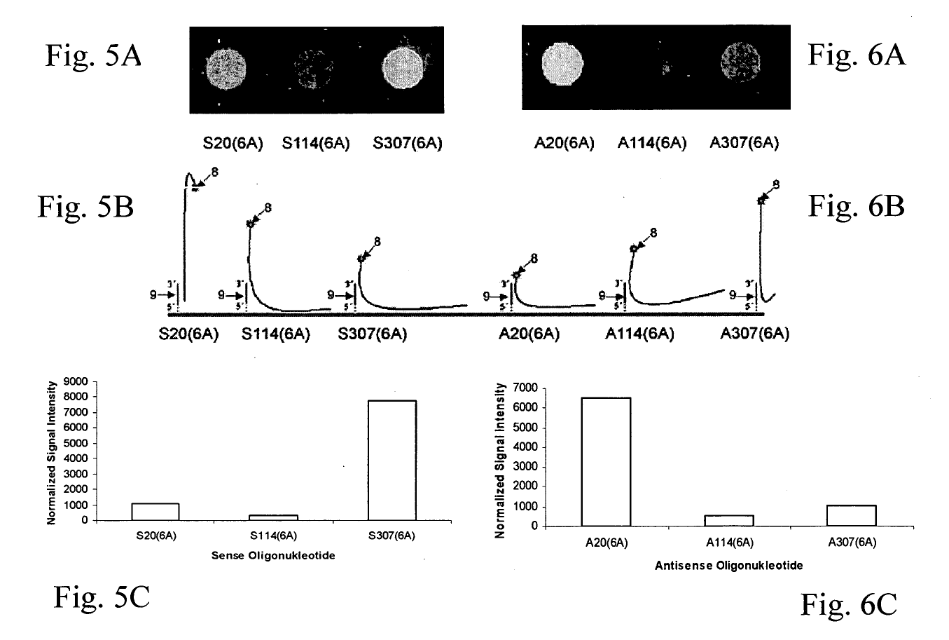

FIG. 5a fluorescence signals of the hybridization of a Cy3-marked antisense strand with sense oligonucleotides

FIG. 5b a schematic representation of the hybrid molecules which lead to the fluorescence signals in FIG. 5a

FIG. 5c a graphical representation of the signal intensities of the fluorescence signals of FIG. 5a

FIG. 6a fluorescence signals of a hybridization of a Cy3-marked sense strand with antisense nucleotides

FIG. 6b a schematic representation of the hybrid molecules which lead to the fluorescence signals of FIG. 6a

FIG. 6c a graphical representation of the signal intensities of the fluorescence signals of FIG. 6a

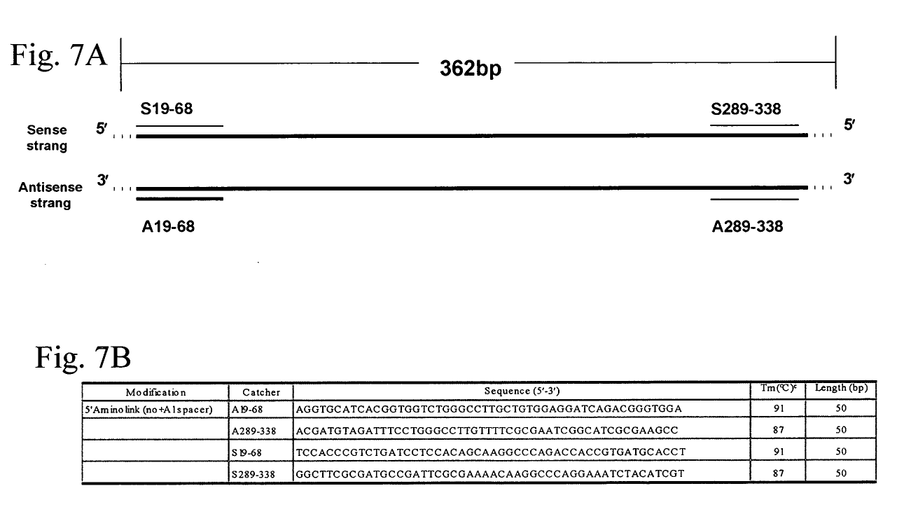

FIG. 7a a schematic representation of 50 mer oligonucleotides according to the embodiments

FIG. 7b a table of the 50 mer oligonucleotides of FIG. 7a

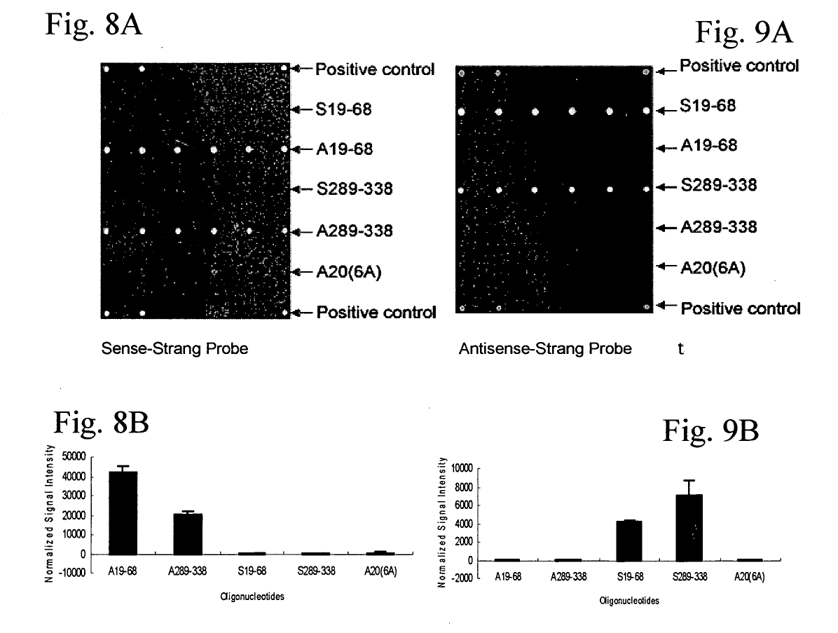

FIG. 8a fluorescence signals of a hybridization of a Cy3-marked sense strand with 50 mer antisense oligonucleotides

FIG. 8b a graphical representation of the signal intensities of the fluorescence signals of FIG. 8a

FIG. 9a fluorescence signals of a hybridization of a Cy3-marked sense strand with 50 mer sense oligonucleotides

FIG 9b a graphical representation of the signal intensities of the fluorescence signals of FIG. 9a

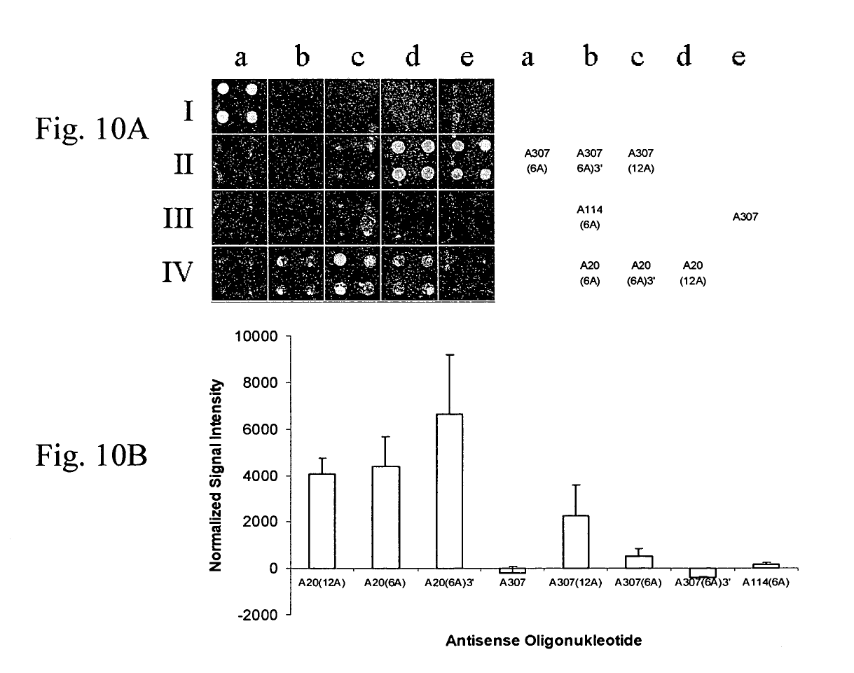

FIG. 10a fluorescence signals of a hybridization of a sense strand, marked with fluorescein-12-dUTP, with short antisense oligonucleotides, wherein the strands are not end-marked, but are uniformly marked internally through the incorporation of fluorescein-12-dUTP

FIG. 10b a graphical representation of the signal intensities of the fluorescence signals of FIG. 10a

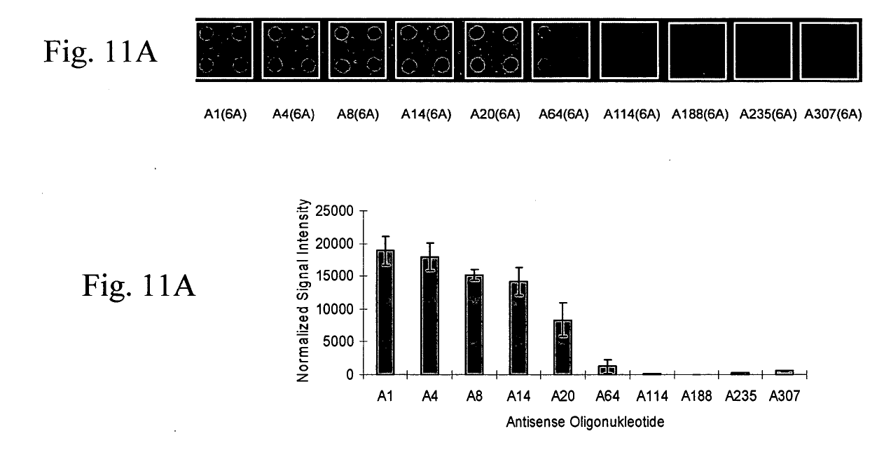

FIG. 11a fluorescence signals of the hybridization of a Cy5-end-marked sense strand with short antisense oligonucleotides as in FIG. 3

FIG. 11b a graphical representation of the signal intensities of the fluorescence signals of FIG. 11a

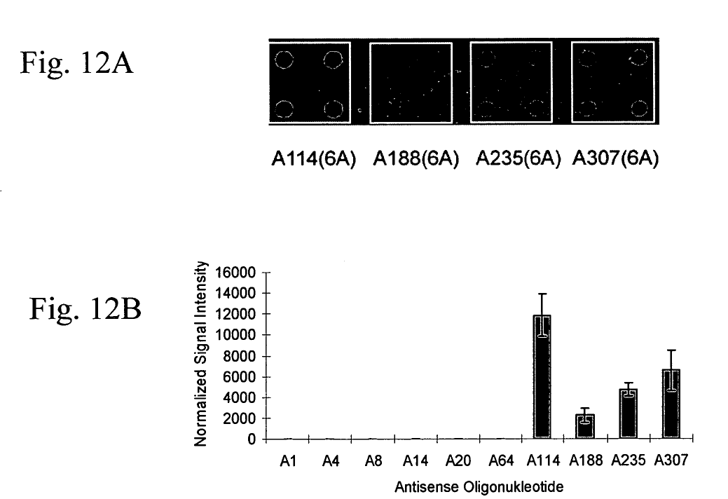

FIG. 12a fluorescence signals of the hybridization of a Cy5-end-marked sense strand, shortened at the 5′ end, with short antisense oligonucleotides as in FIG. 11a, and

FIG. 12b a graphical representation of the signal intensities of the fluorescence signals of FIG. 12a.

FIG. 13 shows the specificities of the catcher molecules of Table 1 which cluster with nitrogenase genes from bacteria of the alpha and beta sub-group of the proteobacteria. The names of the catcher molecules are shown next to the lines which indicate the sequence coverage. The precise sequence coverage is shown by symbols of various types. The nitrogenase genes (nifH, anfH, vnfH) from data banks are identified by their file number.

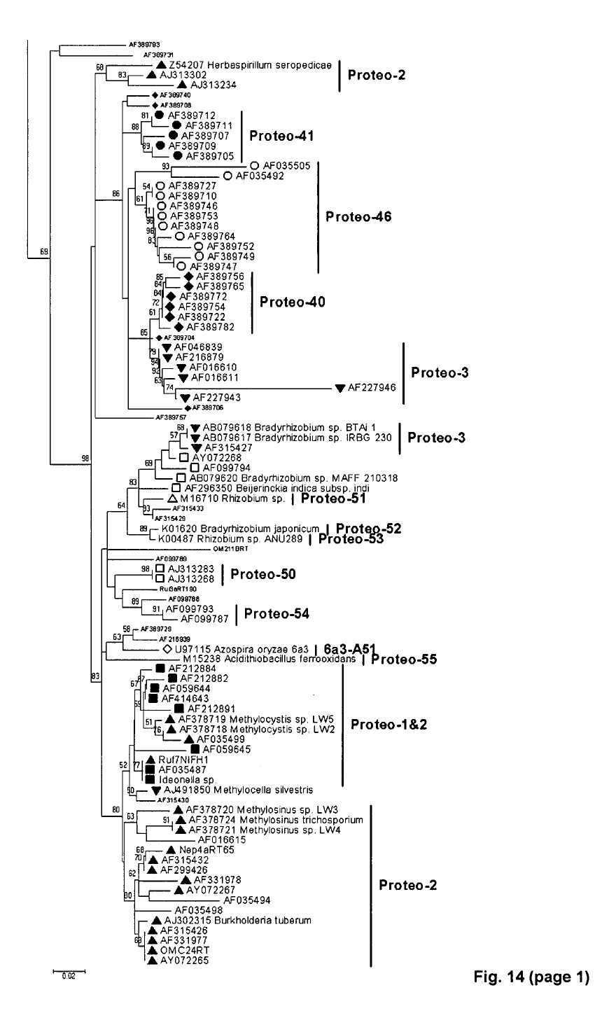

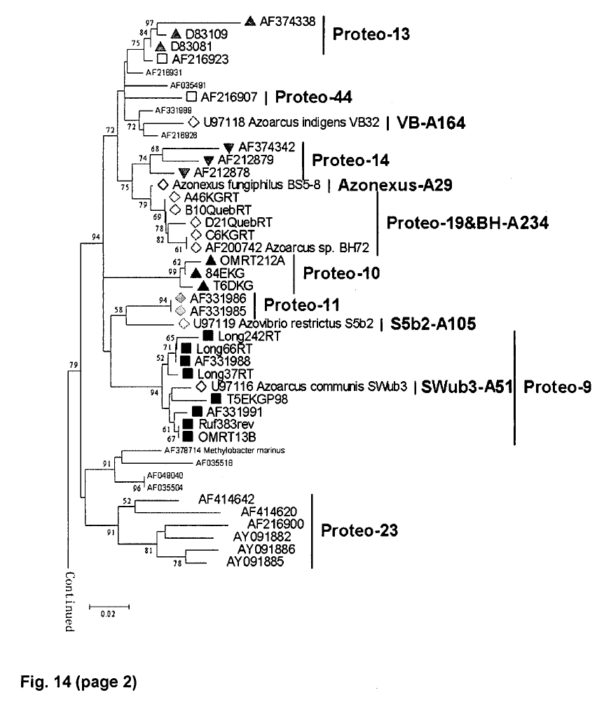

FIG. 14 shows the specificities of the catcher molecules of Table 1 which cluster with nitrogenase genes from bacteria of the beta and gamma sub-group of the proteobacteria. The names of the catcher molecules and the sequence coverage are as shown in FIG. 13.

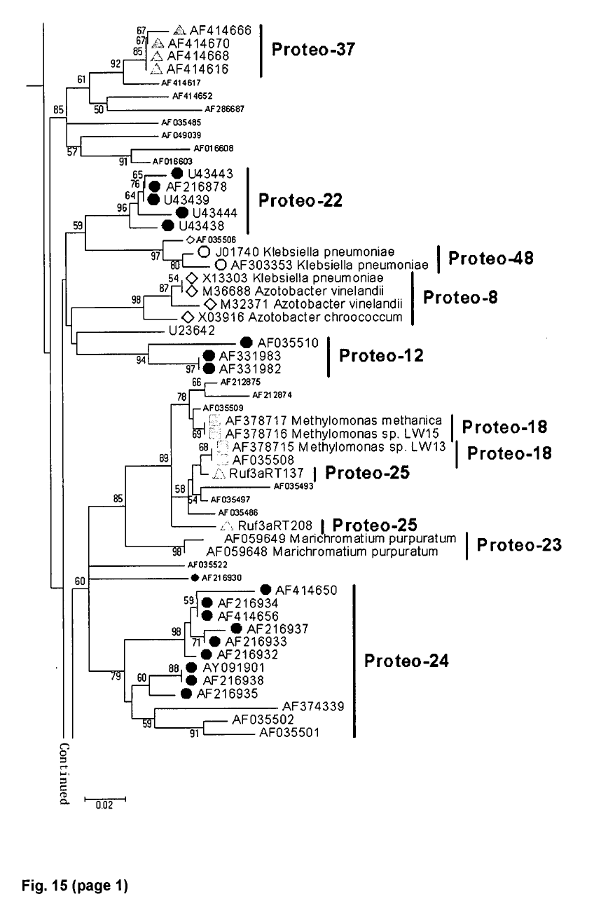

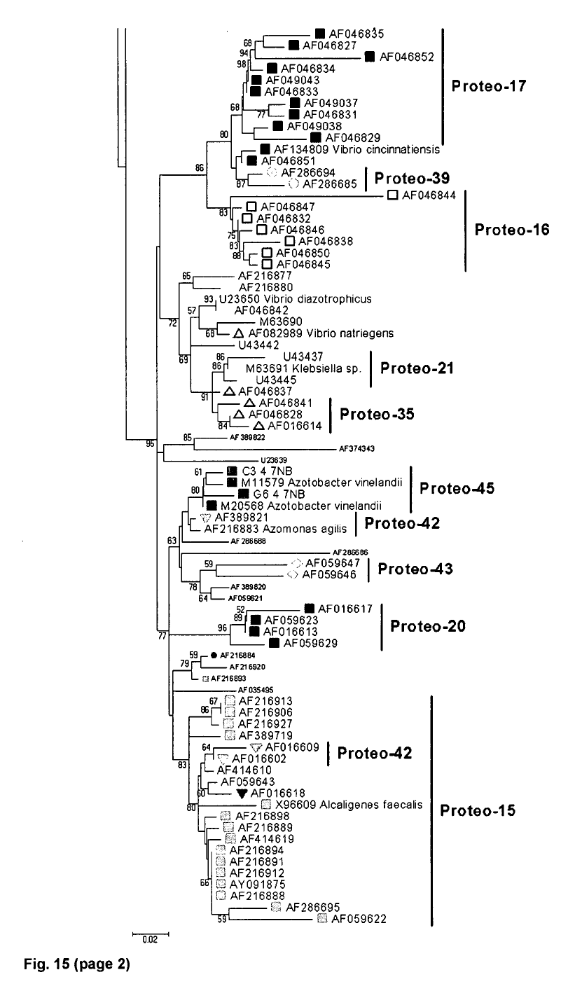

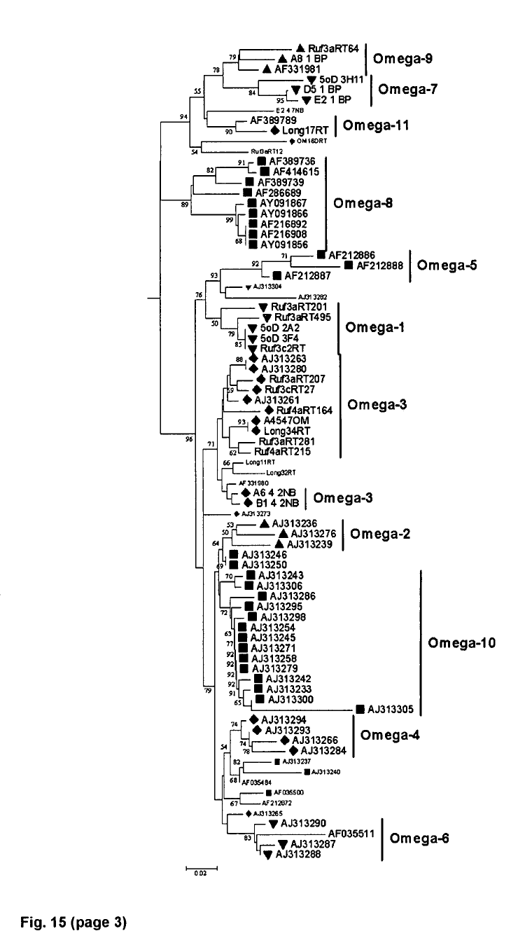

FIG. 15 shows the specificities of the catcher molecules of Table 1 which cluster with nitrogenase genes from bacteria of the omega sub-group of the proteobacteria. The names of the catcher molecules and the sequence coverage are as shown in FIG. 13.

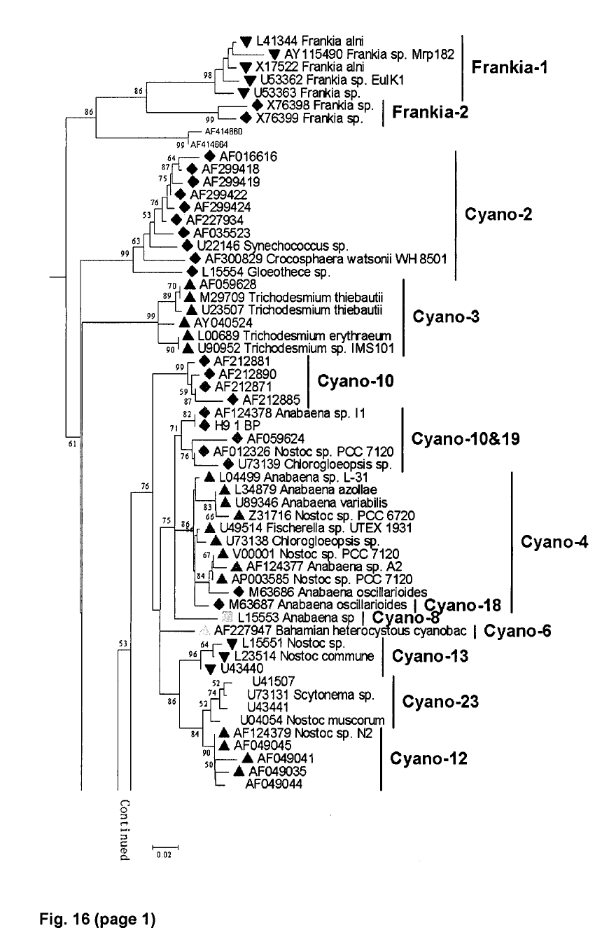

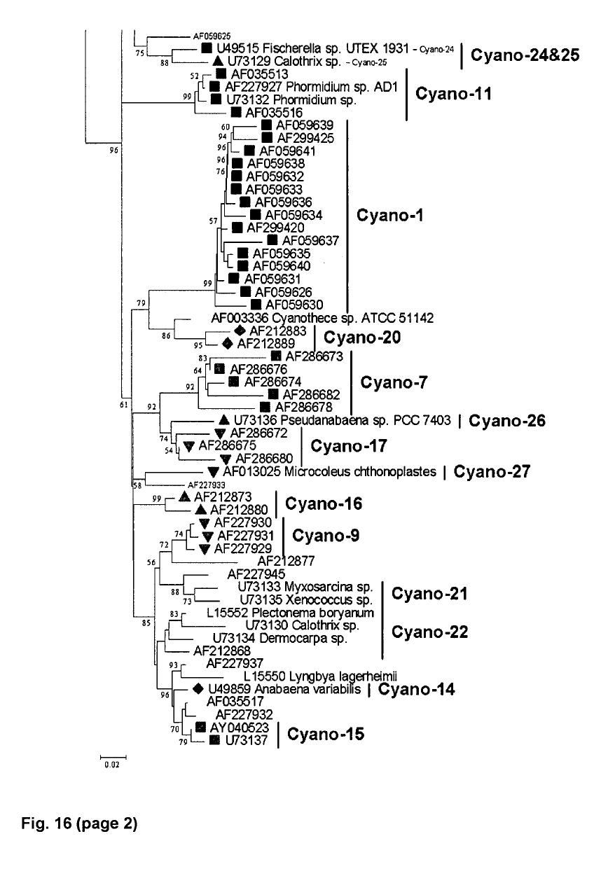

FIG. 16 shows the specificities of the catcher molecules of Table 1 which cluster with nitrogenase genes from frankia, cyanobacteria and similar bacteria. The names of the catcher molecules and the sequence coverage are as shown in FIG. 13.

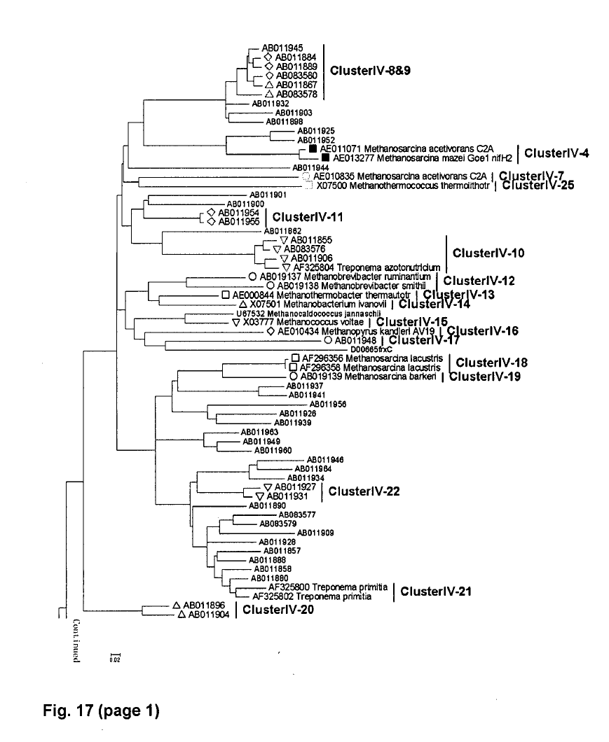

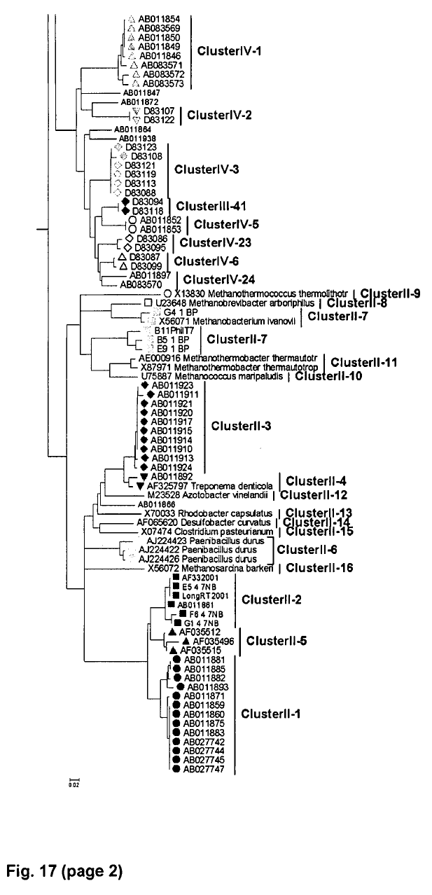

FIG. 17 shows the specificities of the catcher molecules of Table 1 which cluster with nitrogenase genes of clusters II and IV. The names of the catcher molecules and the sequence coverage are as shown in FIG. 13.

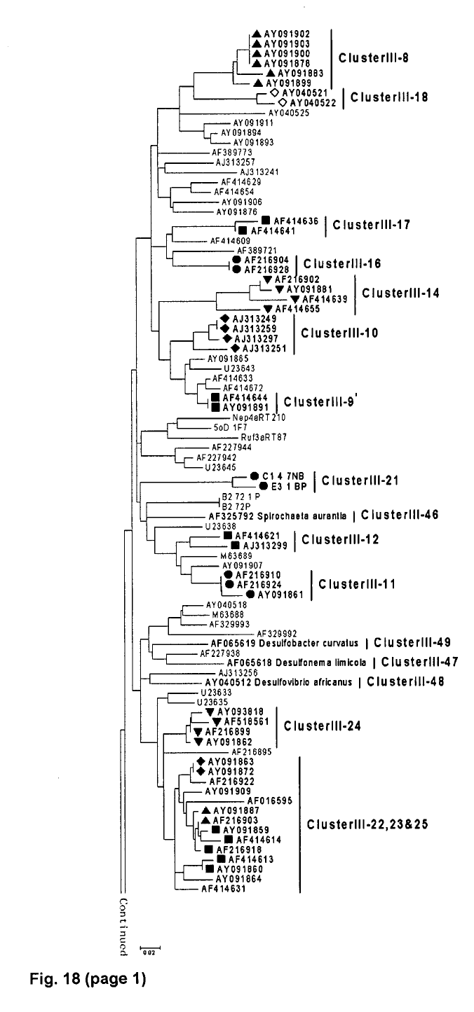

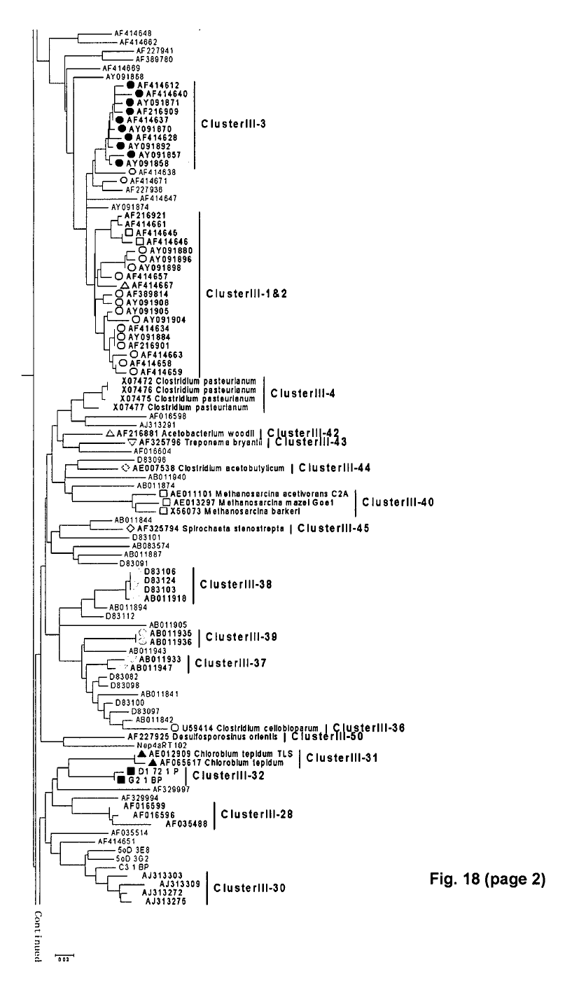

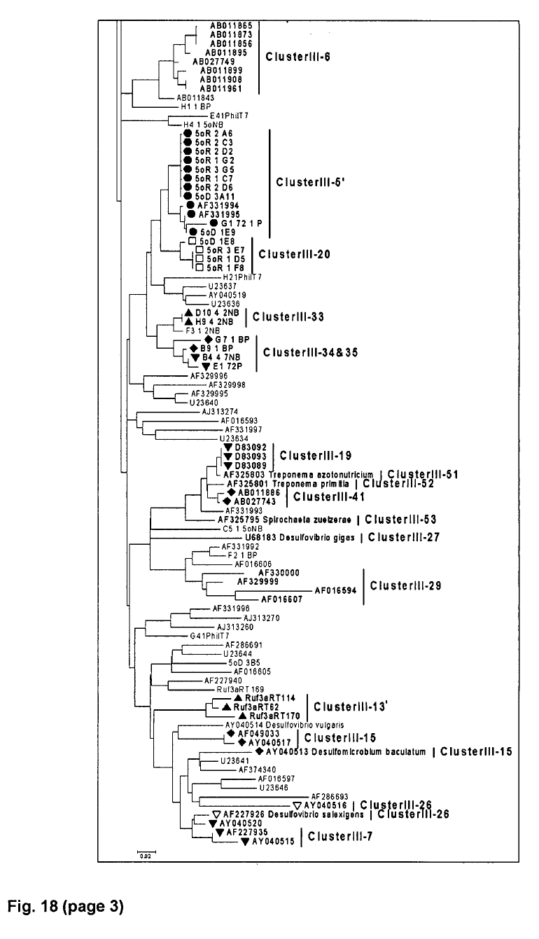

FIG. 18 shows the specificities of the catcher molecules of Table 1 which cluster with nitrogenase genes of cluster III. The names of the catcher molecules and the sequence coverage are as shown in FIG. 13.

The invention is based on the surprising finding that the signal yield from fluorescent marked target molecules forming hybrids or duplexes with catcher sequences in a partial section is greater when the fluorescent marking lies close to the hybrid formed. Unexpectedly this effect is independent of strand and therefore of sequence.

Surprisingly the observed effect is also independent of the chemical nature of the fluorescent marking. The effect occurs with the use of both long (e.g. 50 mer) and short catcher oligonucleotides (e.g. 16-17 mer). Surprisingly this effect is also independent of the glass micro-array surface or coating used to immobilise the catcher oligonucleotides.

The invention may now be better explained with the aid of the following embodiments. The embodiments make it clear that the invention leads to a dramatic improvement in signal yield and in the informative value of micro-array hybridization experiments.

FIG. 1D shows schematically the structure of a duplex or hybrid complex A created by a method according to the invention: at the surface B of a DNA array, for example a glass surface, a catcher sequence or a catcher oligonucleotide D is bound via a spacer C, for example a poly-adenin section of an oligonucleotide. Complexed to the former via a target sequence E is a target molecule F. As may be inferred from FIG. 1D, the target sequence E is only a section or a partial sequence of the target molecule F. In the case of such target molecules with an overhang formed at the duplex, the markings may in principle be provided at any desired point and may also be positioned away from the duplex. Only in the case of such duplexes does the problem on which the invention is based occur, namely that the signal intensities measured, in particular with small sample quantities, are so unstable that no quantitative statement can be made.

A position 1 of the hybrid complex is designated G in FIG. 1D. This is the first base of the hybrid complex A from catcher sequence D and target sequence E. At this point there is a fluorescent marking H. The schematically depicted hybrid complex thus represents an embodiment of the invention, in which the marking H is incorporated within the target sequence F of the target molecule E.

The invention may be better explained with the aid of the following embodiments.

The embodiments which follow show that the invention considerably improves signal intensity in micro-array experiments. This is shown with the aid of specific fluorescent markings. The embodiments demonstrate that the important factor is the position of the marking agent relative to the hybrid to be detected. Here it is completely irrelevant, what specific kind of marking agent is involved. All that matters is the position of the marking agent relative to the hybrid or duplex to be detected, while of course mixed effects involving other factors which may influence the efficiency of the hybridization (e.g. length of the oligonucleotide spacer, steric effects, effects of the secondary structure) occur. The following embodiments should therefore be understood only as explanatory examples; in particular the following embodiments and the experiment also explained do not restrict the teaching of the invention in respect of sequences to be detected, and markings or similar to be used.

### Embodiments

The embodiments are parts of a typical experiment, which was set out as follows:

The target molecule in the typical experiment is a single-stranded DNA, 5′-end-marked or marked with fluorescein-12-dUTP by random labelling, and specifically in the fragment of the nitrogenase gene nifH (Hurek et al., 1995) from the bacterium Azoarcus sp. strain BH72. The fragment was amplified by means of PCR by the primers Zehr-nifH from chromosomal DNA of the strain BH72 (Hurek et al., 2002), with one of the primers being marked with Cy3/Cy5, and the other with biotin. Fluorescent marked single-stranded DNA could thus be isolated from the PCR-product. In connection with the use of fluorescein-12-dUTP for random labelling, only biotinylated primer was used. For all experiments, the primers had the sequences listed in FIG. 1C, primers Z-nifH-f and Z-nifH-r (Zehr and McReynolds, 1989), up to experiments in connection with sequences (Zehr and McReynolds, 1989) following FIG. 11 (primers Z-nifH-f-BH72-Cy5 and Z307-nifH-r-BH72-biotin) and FIG. 12 (primer Z114-nifH-f-BH72-Cy5 and Z307-nifH-r-BH72-biotin):

Biotin-marked strands were separated (Niemayer et al., 1999) by means of streptavadin-coated paramagnetic spheres (Roche). The concentration of the remaining single-stranded DNA was determined by spectral photometry. Before each hybridization, the single-stranded DNA was denatured for 10 minutes at 95° C. and then incubated on ice for at least three minutes.

The oligonucleotides used in this experiment and acting as catcher molecules all bind to the nifH gene fragment of the strain BH72 referred to above. The relevant sequences and their characteristics are set out in table 1, FIG. 1b and table 2, FIG. 7b. A schematic representation of a possible duplex after hybridization is shown in FIG. 1D. Oligonucleotides with either 5′ amino-modifications (amino link c6) or 3′ modifications, some with poly-A spacers of 6-12 nucleotides, were synthesised by the company Thermoelectron (Ulm, Germany).

To conduct hybridization experiments, DNA micro-arrays were created on standard microscopic glass slides made by Menzel of Braunschweig, Germany. Chemicals and solvents came from the company Fluka (Neu-Ulm, Germany). To create the micro-arrays, the glass substrates were cleaned, silylated and activated, as described by Bentas et al (2002). The activated surfaces were used directly for the immobilisation of either 5′ or 3′ amino-modified catcher oligonucleotides by means of covalent binding.

The application of the probes to slide surfaces activated in this way was made using a piezo-driven Spotter Robodrop BIAS, Bremen, Germany) or else a MicroGrid II Compact 400 from the firm of BioRobotics, United Kingdom. The concentration of the oligonucleotides was around 10 μm per ml water. The water used contained 1% glycerol. In each spot of the micro-array approx. 250 pl was applied, corresponding to a spot diameter of around 200 μm.

The slides were incubated overnight at room temperature in a water-saturated atmosphere, in order to effect the covalent binding. Blocking of the micro-arrays was effected by means of 6-amino-1-hexanol (50 mM) and diisopropylethylamine (150 mM) in dimethyl formit after Beier et al (1999). The slides were then washed with deionised, particle-free water, air-dried and stored under N₂ at 4° C.

The hybridization of the target molecules to the probe of the micro-arrays, and washing, took place in a Personal Hyb oven of the company Stratagene, United States of America. Hybridization lasted for 1-16 hours. Unless otherwise stated, hybridization took place at room temperature with 50% formamide, at 46° C. with 50% formamide, and 10 nM single-stranded DNA was used in the process. The hybridization buffer used contained 4×SET, 10×Denhardt's. During hybridization, the slide was covered by a cover glass. After hybridization, washing took place with 2×SET (0.1% SDS) for 5 min. and 1×SET (0.1% SDS) for 10 min. at room temperature, or with 1×(0.1% SDS) for 5 min. and 0.1×SET (0.1% SDS) for 10 min. at 46° C. The dried micro-arrays were analysed at a resolution of 10 μm by a GenePix 4000 Micro-array Scanner from Avon, Union City, Calif., at constant laser strength and constant photomultiplier sensitivity. For this reason the signal intensities determined in the respective embodiments may be compared.

### Embodiment 1

The reverse complementary strand or antisense strand of the nifH gene fragment of strain BH72 referred to above was hybridised with the sense oligonucleotides (catcher molecules) S307 (6A), S114 (6A) and S20 (6A). The antisense strand is shown schematically in FIG. 1A. The Cy5-marking was introduced into the strand by using a Cy5-marked primer to generate the strand. Shown schematically in FIG. 1A are the binding points of the sense oligonucleotide to the antisense strand, with the respective distance of these binding points from the 3′ and 5′ end respectively of the antisense strand also being shown. The antisense strand represents the target molecule, and the sense oligonucleotides represent the catcher oligonucleotides and the catcher sequences, which have been applied to a micro-arrays in the form of probes. The hybridization of these probes by the target molecule leads to different signal intensities in the respective spots on the micro-array, as shown in FIG. 2A. From left to right these spots contain, in each case in pairs, catcher oligonucleotides with the sequence S307 (6A), S114 (6A) and S20 (6A). The antisense strand target molecule is marked only at the 5′ end. Closest to the 5′ end is the binding point and target sequence S307(6A). Hybrids formed at this point are detected in the first two spots shown in FIG. 2A. There the signal intensity is highest. Further removed from the marking is the sequence section S114(6A), which is detected in the next two spots in FIG. 2A. Here the signal is noticeably weaker. Somewhat stronger, but still weak, is the signal given by hybrids into which the binding point S20(6A) enters.

Shown schematically in FIG. 2B are the catcher oligonucleotides 1, 2 and 3, together with the target molecule 4 in the form of hybrid molecules comprised of 1 and 4, 2 and 4, and 3 and 4, and the resultant position of the marking 5 on the target molecule 4 in the respective hybrid. In this case catcher oligonucleotide 1 corresponds to S307(6A), catcher molecule 2 to S114 (6A) and catcher molecule 3 to S20(6A).

The corresponding signal intensities are shown graphically in FIG. 2C. It may be clearly seen that the use of the catcher oligonucleotide S307(6A) or 1 leads to hybrids in which the marking is in close proximity to the hybrid, so that a 3 to 7 times greater signal intensity is obtained as compared to the other hybrids represented. It is also evident that there is an especially large reduction in signal yield when the fragment to be hybridised is bound to the catcher nucleic acid (S114(6A))at a great distance from the marked primer.

### Embodiment 2

This embodiment shows that the effect described in embodiment 1 is independent of strand and therefore of sequence. Cy5-marked counter-strands (sense strand) were hybridised with the corresponding antisense oligonucleotides. The same effect was observed as in example 1 (cf. FIGS. 3A-3C). Shown in FIG. 3A from left to right in each case are four spots with identical catcher oligonucleotides, namely from left to right spots with the catcher oligonucleotides A20(12A), A20(6A), R20(6a)3′, A307, A307(12A), A307(6A), A307(6A)3′, and A114(6A). Shown schematically in FIG. 3B in the same sequence is how the target molecule 7, which has a marking 8 at its end, binds to the relevant oligonucleotide, and the resultant location of the marking relative to the hybrid 9 formed is discernible in FIG. 3B.

As may be seen with the aid of the graphical representation of the signal intensities in FIG. 3C, but is also evident from the brightness of the spots in FIG. 3A, the signal intensities are always at their highest when the marking is in proximity to the respective hybrid formed, and specifically in comparison with the other hybrids by twice to around 4.5 times, in extreme cases well above this level (cf. the values for A307(6A)3′ in FIG. 3C). The designation 6A or 12A (of the catcher oligonucleotides) denotes the length of the respective spacer. This embodiment shows that the difference in length of the spacer, i.e. the spacer between the glass surface of a micro-array and the binding zone of the catcher oligonucleotide, has a comparatively limited effect on signal intensity. Consequently the difference between A20(6A) and A20(12A) is not especially marked, while between A307(12A) and A307(6A) there is virtually no difference at all. A slight reduction of the signal for A20(6A)3′ in comparison with A20(6A) could be due to quenching effects from the close proximity of the fluorescent dye to the glass surface.

In this connection it should be noted that, in micro-array hybridization experiments, false negative results may occur due to a marking lying in an unfavourable position leading to an excessively low signal intensity, as for example in the case of the catcher oligonucleotide A307(6A)3′. Similar steric effects of hybridization have been described by Peplies et al. (2003). This oligonucleotide supplies a signal intensity which is 48 times less than that of A20(6A). The adverse position is far removed from the target sequence.

### Embodiment 3

This embodiment shows that the effect observed in the preceding embodiments may be even further strengthened by greater proximity of the marking to the target sequence. For this purpose an antisense catcher oligonucleotide was used, A1 (6A) in FIG. 11, which is complementary to the primer oligonucleotide with which the end-marked probe was generated by means of PCR. As a result, the fluorescent marking is already positioned at the first nucleotide of the heteroduplex which is formed in hybridization. In this embodiment the fluorescence signal is increased by a factor of 2.3 as compared with a catcher oligonucleotide, A20 (6A), which is shifted by 20 nucleotides. As is evident from FIGS. 11A and 11B, for the intermediate positions (A4 (6A)-A14 (6A)), the signal strengths are progressively weaker than for A1 (6A). In comparison with the most unfavourable position from embodiment 2, A114 (6A), a signal which is as much as 146 times stronger is observed for the most advantageous position A1 (6A). An even further removed but still relatively central position A188 (6A) leads to an even lower signal intensity (factor 357). The embodiment confirms that relatively high signal yields may be obtained when the marking is less than 64 nucleotides from the target sequence forming the heteroduplex; in this embodiment the signal is 15 times weaker.

The positive effect of position is also made clear in FIG. 12. If a shortened marked probe is used (here by 113 nucleotides, so that the primer used for the PCR is complementary to the catcher oligonucleotide A114), a similar position effect is revealed for other catcher oligonucleotides. Oligonucleotide A114 (6A), which in FIG. 11 with a central position gave only low signal strength levels (130 rel. intensity), shows in FIG. 12 as end-placed catcher oligonucleotide high signal strength (11,800 rel. intensity). In this example too, a sharp drop in signal intensity (factor 5.2) is to be observed when the marking is at a greater distance (e.g. 74 nucleotides for A 188 (A6), FIG. 12A, B) from the formed hybrid.

### Embodiment 4

This embodiment shows that the effect observed in the preceding embodiments is independent of the chemical nature of the marking. The same experiments as in embodiment 2 were conducted with a Cy3-marked sense strand instead of a Cy5-marked sense strand, and supplied substantially the same results as described in example 2. These results are set out in FIGS. 4A-4C, in which the markings in FIG. 4B are provided with reference number 10, and the accordingly marked target molecule with reference number 11. Shown from left to right in FIG. 4A in each case are four spots in which catcher oligonucleotides as described in embodiment 2 are present. FIG. 4B shows from left to right in each case schematically how the respective catcher oligonucleotide is hybridised with the target molecule, and in which position the marking is to be found in each case, relative to the hybrid formed. FIG. 4C shows the signal intensities of the spots shown in FIG. 4A, in the same sequence.

As already indicated, the results correspond substantially to those discussed in embodiment 2. The fact that a different marking leads to substantially the same results confirms that it does not matter what type of fluorescent marking is used in implementing the invention.

### Embodiment 5

The experiment described in embodiment 1 was repeated with Cy3-marked sense strand and Cy3-marked antisense strand. The results in the case of the Cy3-marked sense strand are shown in FIGS. 5A-5C. The results of the experiment conducted with the Cy3-marked antisense strand are depicted in FIGS. 6A-6C. In each case the signal intensity is greatest where the marking 8 is in direct proximity to the respectively formed hybrid 9. When the Cy3-marked sense strand is used as target molecule this is the case in the spot with the catcher sequence for S307 (6A), and for the Cy3-marked antisense strand it is in the spot with the catcher sequence for A20(6A) (see FIGS. 5A-5C, FIGS. 6A-6C).

These experiments comparable with embodiment 1 confirm that both the Cy3-marked sense strand and also the Cy3-marked antisense strand are detectable with high signal yields when the hybrids formed with catcher sequences have the marking in direct proximity. In the case of probes A20(6A) or S307(6A), signal intensity is increased by a factor of 22 as compared with other hybrids.

### Embodiment 6

This embodiment confirms that the invention also functions when longer oligonucleotides are used as catcher molecules. In this case 50 mer oligonucleotides were used, binding in each case at the outer ends of the target molecule. Cy3-marked sense strand shows the stronger signal when the marking lies close to the duplex (A19-68). In this case signal intensity was increased by a factor of 2 over the other signals.

The designation and sequences of the 50 mer oligonucleotide catchers and target sequences used may be taken from table 2 in FIG. 7B. The position of the relevant binding points on the sense or antisense strand of the 362 bp long nifH gene fragment are shown schematically in FIG. 7A.

Shown in FIG. 8A are the spots of a corresponding micro-array, with in each case six identical spots in one row. The target sequences or catcher sequences detectable in the respective spots are plotted on the right-hand side of the represented micro-array surface in the representation of FIG. 8A. In FIG. 8B, the correspondingly determined signal intensities are shown in the form of a graphical presentation.

The same experiment was conducted with Cy3-marked antisense strand. In this case too, the strongest signal is obtained when the marking is close to the duplex (S289-338), see FIGS. 9A, 9B. Here too the signal intensity with use of a method according to the invention was increased by a factor of 2 over other signal intensities, at any rate distinctly improved, as shown by FIG. 9b for the sequence S289-338.

### Embodiment 7

This embodiment confirms that other marking strategies may also be used to produce target molecules. Using unmarked PCR primer and the random incorporation of fluorescein-12-dUTP (“random labelling”), a suitably marked sense strand was created, and hybridised with short antisense oligonucleotides. On the left of FIG. 10A, in a grid comprised of rows and columns, in each case four probe spots or spots are shown. The rows are numbered consecutively from I to IV, and the columns are designated a-e. Next to this on the right is a schematic reproduction of the same grid, with the designation of the catcher oligonucleotides located in the respective spots entered in the corresponding grid areas. Thus for example field IIa contains spots with the catcher oligonucleotides A307(6A) etc. In FIG. 10E the signal intensities determined for the respective spots are presented graphically. The highest are the signal intensities in spot A20(6A)3′, followed by spot A20(6A) and A20(12A).

Table I in FIG. 1B discloses that the catcher oligonucleotide A20(6A)3′ binds at four positions to T and U respectively, whereas the catcher oligonucleotide 307 binds to T and U respectively at 0 positions, so that there is a higher incorporation rate of fluorescent marked nucleotides in the A20 target sequence. Through fluorescent marking on these bases there is in consequence a greater fluorescent intensity in the duplex A 20 than at A307, resulting in a noticeably higher signal intensity (see FIG. 10B).

In addition to the glass slides described above, commercial supports for micro-arrays were also used, for example aldehyde slides and amine slides, plus slides from the company Genetics, QMT® aldehyde slides from Peqlab, and Pan ® amine slides from MWG Biotech. With these micro-arrays the same results were obtained as described above with the aid of embodiments 1-7.

This confirms that the present invention may be used in conjunction with any type of micro-array.

It has been shown above, with the aid of target sequence marked according to the invention and applied to DNA arrays in solution, that the invention is independent of strand and therefore of sequence.

Within the scope of the invention, this procedure may easily be reversed, i.e. target molecules to be analysed may be provided on an array, to which catcher molecules in solution and marked according to the invention are added, so that duplexes or complexes with high signal intensities are created. In other words, the term “target molecule” is to be understood as being interchangeable with the term “catcher molecule” and vice-versa. At the same time the term “target sequence” should then be understood as being interchangeable with the term “catcher sequence” and vice-versa.

Also, within the scope of the invention, the entire ligand binding reaction may in principle be effected in solution.

TABLE 1

Sequences of catcher oligonucleotides for nitrogenase
gene detection. Proximity to the hybrid is shown in
the table (_{”}Position“ column).

| | | | | | | |
|---|---|---|---|---|---|---|
| | SEQ | | | | | |
| | ID | | | Length | | |
| Sample^{a} | NO | Sequence (5′-3′)^{b} | Tm^{c} | (nt) | GC% | Position^{d} |

| | | | | | | |
|---|---|---|---|---|---|---|
| Proteo-1 | 1 | GACAAGATGGTGTCCTGAG | 67 | 19 | 52 | 29-47 |
| Proteo-2 | 2 | GACAGGATGGTGTCCTG | 66 | 17 | 58 | 31-47 |
| Proteo-3 | 3 | GGCTCAGGATGGTGTCC | 68 | 17 | 64 | 33-49 |
| Proteo-4 | 4 | AGGACGGTGTCCTGGG | 68 | 16 | 68 | 29-44 |
| Proteo-5 | 5 | TTCCGCGGCAAGGGAGA | 68 | 17 | 64 | 44-60 |
| Proteo-6 | 6 | GTGTCCTGCAGCTTGGT | 66 | 17 | 58 | 22-38 |
| Proteo-7 | 7 | AGCCGATCTTGAGAACGTC | 67 | 19 | 52 | 91-109 |
| Proteo-8 | 8 | AGCCGATCTGCAGCACGT | 69 | 18 | 61 | 92-109 |
| Proteo-9 | 9 | CAGTTCCATCACGGAGTTC | 67 | 19 | 52 | 33-51 |
| Proteo-10 | 10 | TGCATGACGCTGGTTTGC | 67 | 18 | 55 | 30-47 |
| Proteo-11 | 11 | TACCCGATGGCCAGCACAT | 69 | 19 | 57 | 92-110 |
| Proteo-12 | 12 | ATGACGGTGTTCTGCATCTT | 66 | 20 | 45 | 25-44 |
| Proteo-13 | 13 | CACGGTCGTTTGCGCCTT | 69 | 18 | 61 | 25-42 |
| Proteo-14 | 14 | TCTGAGCCTTTGAGTGCAG | 67 | 19 | 52 | 16-34 |
| Proteo-15 | 15 | TTGATGTCGCCGTAGCCG | 69 | 18 | 61 | 105-122 |
| Proteo-16 | 16 | TTTAATGCCGCTGTAACCAGT | 66 | 21 | 42 | 103-123 |
| Proteo-17 | 17 | CCAGTCAGTAGTACATCTTCTA | 66 | 22 | 40 | 86-107 |
| Proteo-18 | 18 | TTTTGCGCTTTCGCATGCAG | 68 | 20 | 50 | 16-35 |
| Proteo-19 | 19 | CTTGCTGTGGAGGATCAG | 67 | 18 | 55 | 10-27 |
| Proteo-20 | 20 | CTCCATTATTGTGTTTTGCGC | 66 | 21 | 42 | 28-48 |
| Proteo-21 | 21 | GTGTTTTGCGCTTTAGAGTG | 66 | 20 | 45 | 19-38 |
| Proteo-22 | 22 | GGAAGTTAATCGCTGTGATAAC | 66 | 20 | 40 | 175-196 |
| Proteo-23 | 23 | ATGGTCTCCTGGGCCTT | 66 | 17 | 58 | 25-41 |
| Proteo-24 | 24 | CACTTGACGTCGCCGTA | 66 | 17 | 58 | 109-125 |
| Proteo-25 | 25 | TTCCAAATCTTCAACGCTACC | 66 | 21 | 42 | 64-84 |
| Proteo-26 | 26 | GTGACAGCACCGACGTC | 67 | 18 | 64 | 33-49 |
| Proteo-27 | 27 | ACGGTCTGCTGGGCTTT | 66 | 17 | 58 | 25-41 |
| Proteo-28 | 28 | ATGCAGGACCGTGTCCTG | 69 | 18 | 61 | 31-48 |
| Proteo-29 | 29 | AGATGCAGAACCGTGTCCT | 67 | 19 | 52 | 32-50 |
| Proteo-30 | 30 | GAACCTTCGGTTGCCGC | 68 | 17 | 64 | 52-68 |
| Proteo-31 | 31 | GCGGCGAGATGCAGAAC | 68 | 17 | 64 | 40-56 |
| Proteo-32 | 32 | ATCCAGGACGGTATCCTG | 67 | 18 | 55 | 31-48 |
| Proteo-33 | 33 | GATGTCCTGATAGCCGAC | 67 | 18 | 55 | 103-120 |
| Proteo-34 | 34 | TTGATGTCCTTGAAGCCGA | 65 | 19 | 47 | 104-122 |
| Proteo-35 | 35 | GCCTGATTCAACACAACGAAC | 68 | 21 | 47 | 118-138 |
| Proteo-36 | 36 | CCAGCGAAAGGACGGTG | 68 | 17 | 64 | 36-52 |
| Proteo-37 | 37 | AATATCTTTAAAACCGGCTTTCAG | 66 | 24 | 33 | 97-120 |
| Proteo-38 | 38 | GTGTCCTGCATCTTGGTG | 67 | 18 | 55 | 21-38 |
| Proteo-39 | 39 | GGTGTTTTGCATTTTAGTGTG | 64 | 21 | 38 | 19-39 |
| Proteo-40 | 40 | CCAGCGAGAGGATGGTG | 68 | 17 | 64 | 36-52 |
| Proteo-41 | 41 | CTTGGCATGCAGCATCAG | 67 | 18 | 55 | 10-27 |
| Proteo-42 | 42 | ATGCAGAATCAGTCGAGTAGA | 66 | 21 | 42 | 1-21 |
| Proteo-43 | 43 | GTGTTCTGTGCTTTAGAGTGAAG | 69 | 23 | 43 | 16-38 |
| Proteo-44 | 44 | GTGCATTACAGTAGTCTG | 62 | 18 | 44 | 31-48 |
| Proteo-45 | 45 | GTAGCCAGCCTTCAGCAC | 69 | 18 | 61 | 94-111 |
| Proteo-46 | 46 | AGGCTGAGGATCGTGTCC | 69 | 18 | 61 | 33-50 |
| Proteo-47 | 47 | CAGCAGCAAGGTGCAGC | 68 | 17 | 64 | 42-58 |
| Proteo-48 | 48 | GCCATCTCCATAATGGTGT | 65 | 19 | 47 | 35-53 |
| Proteo-49 | 49 | CCTTGCAGCCGAGGATC | 68 | 17 | 64 | 12-28 |
| Proteo-50 | 50 | AGGCTGAGGATGGTGTC | 66 | 17 | 58 | 34-50 |
| Proteo-51 | 51 | CTCGAGGTCTTCGACGCT | 69 | 18 | 61 | 67-84 |
| Proteo-52 | 52 | CTCGCGGCAAGACTCAAA | 67 | 18 | 55 | 42-59 |
| Proteo-53 | 53 | CTCGCTGCAAGGCTCAAA | 67 | 18 | 55 | 42-59 |
| Proteo-54 | 54 | CGTGTAGGATCAGCCGTGT | 69 | 19 | 57 | 4-22 |
| Proteo-55 | 55 | AGACTAAGCACGGTGTCTTG | 68 | 20 | 50 | 31-50 |
| Omega-1 | 56 | TGTCTGAGCCTTGGCATGA | 67 | 19 | 52 | 18-36 |
| Omega-2 | 57 | CTTCATAACGTCCTTCAGTTC | 66 | 21 | 42 | 79-99 |
| Omega-3 | 58 | GGTCCATAACCGTTGACTG | 67 | 19 | 52 | 31-49 |
| Omega-4 | 59 | CCATTACCGTGTTCTGTGC | 67 | 19 | 52 | 28-46 |
| Omega-5 | 60 | GGTGTCGCCATAGCCGA | 68 | 17 | 64 | 104-120 |
| Omega-6 | 61 | CAACCTTCATCACGGATTCG | 68 | 20 | 50 | 87-106 |
| Omega-7 | 62 | CGGATGAGGTCCATCAC | 66 | 17 | 58 | 40-56 |
| Omega-8 | 63 | GAACCTTGTCCATAACCGT | 64 | 19 | 47 | 37-55 |
| Omega-9 | 64 | CTCGCGGATCAGGTCCAT | 69 | 18 | 61 | 43-60 |
| Omega-10 | 65 | GACCTTCATGACATCTTCCAG | 68 | 21 | 47 | 85-105 |
| omega-11 | 66 | ACGTCCGAGAGCTCCAGA | 69 | 18 | 61 | 78-95 |
| Cyano-1 | 67 | CAGTGGTTTGTGCTTTACA | 63 | 19 | 42 | 22-40 |
| Cyano-2 | 68 | AGTGAAGAACTGTTACCTGTGC | 68 | 22 | 45 | 28-49 |
| Cyano-3 | 69 | GTTTGAGCTTTAGCGTTTAAGATTA | 66 | 25 | 32 | 11-35 |
| Cyano-4 | 70 | AAACCGGTCAACATTACTTCGTG | 69 | 23 | 43 | 88-110 |
| Cyano-5 | 71 | GCACCTGGTTTACATACATC | 66 | 20 | 45 | 91-110 |
| Cyano-6 | 72 | GCTTAGTACGGTTGTTTGTG | 66 | 20 | 45 | 29-48 |
| Cyano-7 | 73 | TCTACACATCGGATACCTTTGTA | 67 | 23 | 39 | 109-131 |
| Cyano-8 | 74 | CTGCACCTTTTTCAGCAGC | 67 | 19 | 52 | 52-70 |
| Cyano-9 | 75 | TACAGTGGAGCATTAAGCGAG | 68 | 21 | 47 | 5-25 |
| Cyano-10 | 76 | CAGCCAAGTGAAGAACGGT | 67 | 19 | 52 | 37-55 |
| Cyano-11 | 77 | CACTTAACGCCGCGATAGC | 69 | 19 | 57 | 107-125 |
| Cyano-12 | 78 | ATTACTTCTTCGAGTTCAATATCTT | 65 | 25 | 28 | 74-98 |
| Cyano-13 | 79 | ACGAACGTTACGGAAACC | 64 | 18 | 50 | 106-123 |
| Cyano-14 | 80 | CTAAGTGGAGAATGGTAGTTTG | 66 | 22 | 40 | 31-52 |
| Cyano-15 | 81 | GGTGAAGAATCGTGGTTTG | 65 | 19 | 47 | 31-49 |
| Cyano-16 | 82 | GGTGTAGAATTGTGGTTTGG | 66 | 20 | 45 | 30-49 |
| Cyano-17 | 83 | AGCTAGGTGCAGAACTGTG | 67 | 19 | 52 | 36-54 |
| Cyano-18 | 84 | GCAGCCAAGGAAAGAACG | 67 | 18 | 55 | 39-56 |
| Cyano-19 | 85 | CTTCACACCACGGAAGCC | 69 | 18 | 61 | 106-123 |
| Cyano-20 | 86 | CGAGCAATACTTCCGAGAGT | 68 | 20 | 50 | 84-103 |
| Cyano-21 | 87 | ACGTTGTTATAGCCAGTGAGTA | 66 | 22 | 40 | 98-119 |
| Cyano-22 | 88 | GTGCAGAATGGTGGTTTG | 64 | 18 | 50 | 31-48 |
| Cyano-23 | 89 | CAGCAGCCAATTGGAGAAC | 67 | 19 | 52 | 40-58 |
| Cyano-24 | 90 | CTTTTCTGCTGCCAGGTG | 67 | 18 | 55 | 46-63 |
| Cyano-25 | 91 | AGCTTTACAGTGAAGAATCAAGC | 67 | 23 | 39 | 8-30 |
| Cyano-26 | 92 | GAGCTTCATCAAGTTCCAC | 65 | 19 | 47 | 79-97 |
| Cyano-27 | 93 | CTGGATGCCAGCGTAGC | 68 | 17 | 64 | 107-123 |
| Frankia-1 | 94 | ATGCCCCACTGGCCCTC | 71 | 17 | 70 | 103-119 |
| Frankia-2 | 95 | GCCTTCTCGATGACGGTG | 69 | 18 | 61 | 36-53 |
| ClusterII-1 | 96 | CCCAGAATCATGCGGGTA | 67 | 18 | 55 | 3-20 |
| ClusterII-2 | 97 | GTTCATTCCGCCTAAAATCATAC | 67 | 23 | 39 | 8-30 |
| ClusterII-3 | 98 | GTTTTGATGACCTTCTCGTTG | 66 | 21 | 42 | 81-101 |
| ClusterII-4 | 99 | ACATCCATCAGGGTTTCCTG | 68 | 20 | 50 | 31-50 |
| ClusterII-5 | 100 | TTGATTCATCCCTCCCAAAA | 64 | 20 | 40 | 14-33 |
| ClusterII-6 | 101 | TATCCATCAATGTTTCTTGCGG | 66 | 22 | 40 | 28-49 |
| ClusterII-7 | 102 | CCATCATGGTGGTCTGCA | 67 | 18 | 55 | 29-46 |
| ClusterII-8 | 103 | GCATTTTACCTCTAAGAATCATAC | 66 | 24 | 33 | 8-31 |
| ClusterII-9 | 104 | GTTTTCCGTGGAGAATCATTC | 66 | 21 | 42 | 8-28 |
| ClusterII-10 | 105 | CCGTGCAAGATTAACCTTG | 65 | 19 | 47 | 5-23 |
| ClusterII-11 | 106 | GTTGTCTGCATTTTACCGC | 65 | 19 | 47 | 20-38 |
| ClusterII-12 | 107 | GGTCTCCTCAGGTTTGC | 66 | 17 | 58 | 23-39 |
| ClusterII-13 | 108 | ATCACCGTCTGCTGCGG | 68 | 17 | 64 | 28-44 |
| ClusterII-14 | 109 | CCAGGATCAGCCGATTTGA | 67 | 19 | 52 | 1-19 |
| ClusterII-15 | 110 | CACCAAGGATAAGACGAGTT | 66 | 20 | 45 | 3-22 |
| ClusterII-16 | 111 | CGAGCACAAGACGAGTACA | 67 | 19 | 52 | 1-19 |
| ClusterIII-1 | 112 | CCTTCTTCGCGGAGCGT | 68 | 17 | 64 | 49-65 |
| ClusterIII-2 | 113 | ACTCGGTGCACCGGCAA | 68 | 17 | 64 | 111-127 |
| ClusterIII-3 | 114 | CCTTCCTCGCGGAGTGT | 68 | 17 | 64 | 49-65 |
| ClusterIII-4 | 115 | GAAGTGATTATTCCTCTTCC | 64 | 20 | 40 | 160-179 |
| ClusterIII-5 | 116 | CGCCTAAGAGAAGACGAGT | 67 | 19 | 52 | 4-22 |
| ClusterIII-6 | 117 | TCGTCGCGAAGGGTATCCA | 69 | 19 | 57 | 44-62 |
| ClusterIII-7 | 118 | CCGCCTTTACGGATATC | 63 | 17 | 52 | 85-101 |
| ClusterIII-8 | 119 | CCGCAAGGTCCACGTC | 68 | 16 | 68 | 70-85 |
| ClusterIII-9′ | 120 | CTTCCCGGCGGATGTC | 68 | 16 | 68 | 85-100 |
| ClusterIII-10 | 121 | GCAGCGTATCCAATACGGT | 67 | 19 | 52 | 37-55 |
| ClusterIII-11 | 122 | GTCTTCCCCTTCCGACC | 68 | 17 | 64 | 56-72 |
| ClusterIII-12 | 123 | TCTTCCAGATCCACGTCTTC | 68 | 20 | 50 | 67-86 |
| ClusterIII-13′ | 124 | CTTTTCTGCGCAAGACCAC | 67 | 19 | 52 | 20-38 |
| ClusterIII-14 | 125 | ACCCTGTCCGGCGGATA | 68 | 17 | 64 | 87-103 |
| ClusterIII-15 | 126 | GGGTATCCAGAACGGACTT | 67 | 19 | 52 | 34-52 |
| ClusterIII-16 | 127 | ACGGTCCGTTGACTCAAAC | 67 | 19 | 52 | 23-41 |
| ClusterIII-17 | 128 | GAGCCAAGCCATGCAACA | 67 | 18 | 55 | 14-31 |
| ClusterIII-18 | 129 | GTATCTAACACACTCTTTTGGT | 65 | 22 | 36 | 29-50 |
| ClusterIII-19 | 130 | GTTCTGAGTGTATCCAACAC | 66 | 20 | 45 | 40-59 |
| ClusterIII-20 | 131 | CCAAGGAGAAGACGGGTG | 69 | 18 | 61 | 3-20 |
| ClusterIII-21 | 132 | GTCCAAGACGGTGCTTTG | 67 | 18 | 55 | 31-48 |
| ClusterIII-22 | 133 | TCGAGAAGATTGATGGAGG | 65 | 19 | 47 | 176-194 |
| ClusterIII-23 | 134 | TATCCAGGACAGTGCGCT | 67 | 18 | 55 | 32-49 |
| ClusterIII-24 | 135 | TCCAAAACGGTCCGCTG | 66 | 17 | 58 | 31-47 |
| ClusterIII-25 | 136 | CCGAAGCCCTGTTTGCG | 68 | 17 | 64 | 91-107 |
| ClusterIII-26 | 137 | AACCGGGTTTACGGATATC | 65 | 19 | 47 | 85-103 |
| ClusterIII-27 | 138 | AAAGCCGGGCGACACGA | 68 | 17 | 64 | 89-105 |
| ClusterIII-28 | 139 | CACCTAAAAGTAGACGTGTTGA | 66 | 22 | 40 | 1-22 |
| ClusterIII-29 | 140 | CCAACAACAATCGGGTTGA | 65 | 15 | 47 | 1-19 |
| ClusterIII-30 | 141 | CACAGCGAACACCTTTAAAAC | 66 | 21 | 42 | 101-121 |
| ClusterIII-31 | 142 | CGGTTTTCTGCTGAAGACC | 67 | 19 | 52 | 22-40 |
| ClusterIII-32 | 143 | GTCTTTTGTGCTAAACCACC | 66 | 20 | 45 | 19-38 |
| ClusterIII-33 | 144 | CACCTTCCGCGAGTGTAT | 67 | 18 | 55 | 47-64 |
| ClusterIII-34 | 145 | AGTACGGTTTTCTGGGCTAA | 66 | 20 | 45 | 25-44 |
| ClusterIII-35 | 146 | CTCCCAATAAGAGACGGG | 67 | 18 | 55 | 5-22 |
| ClusterIII-36 | 147 | GTATCCTACCTTCAGAACCG | 68 | 20 | 50 | 86-105 |
| ClusterIII-37 | 148 | TCAATGTCATCGCCTTCTTC | 66 | 20 | 45 | 58-77 |
| ClusterIII-38 | 149 | ATGCCGGAAAAGCCGGG | 68 | 17 | 64 | 97-113 |
| ClusterIII-39 | 150 | CCAGCTCTATGTCGTCGC | 69 | 18 | 61 | 65-82 |
| ClusterIII-40 | 151 | GTCTTCTGGTTCAGGCC | 66 | 17 | 58 | 22-38 |
| ClusterIII-41 | 152 | CGTATCAAGCACCGTTTTC | 65 | 19 | 47 | 33-51 |
| ClusterIII-42 | 153 | CAAAATGGCATCCAATTCAATTTC | 66 | 24 | 33 | 70-93 |
| ClusterIII-43 | 154 | CATGATACGGTCGAGTTCAAC | 68 | 21 | 47 | 73-93 |
| ClusterIII-44 | 155 | CTGAGCTAATCCTCCTAAAAG | 66 | 21 | 42 | 13-33 |
| ClusterIII-45 | 156 | GGTGAAGACCACCAAGAAG | 67 | 19 | 52 | 13-31 |
| ClusterIII-46 | 157 | TGCTGGAGTCCTCCCAAC | 69 | 18 | 61 | 15-32 |
| ClusterIII-47 | 158 | CAAGTTTTCGCACATCATCCAG | 68 | 22 | 45 | 79-99 |
| ClusterIII-48 | 159 | GCCGAGCTTGATGATGTC | 67 | 18 | 55 | 85-102 |
| ClusterIII-49 | 160 | CGACTTTTCTAACGTCTTCAAG | 66 | 22 | 40 | 79-100 |
| ClusterIII-50 | 161 | GTACGGTCTTTTGGCTCAAAC | 68 | 21 | 47 | 23-43 |
| ClusterIII-51 | 162 | CGGTTCGCAATGTATCCAG | 67 | 19 | 52 | 43-61 |
| ClusterIII-52 | 163 | CAGGCCGTTCAGCAAAAG | 67 | 18 | 55 | 10-27 |
| ClusterIII-53 | 164 | GCCTCCCAGAAGCAAAC | 66 | 17 | 58 | 8-24 |
| ClusterIV-1 | 165 | CCCTGATGGCGTCAAGC | 68 | 17 | 64 | 42-58 |
| ClusterIV-2 | 166 | AGTACCGTTTCCTGGTGCA | 67 | 19 | 52 | 26-44 |
| ClusterIV-3 | 167 | CGGTTCGGTTTTGCCGTC | 69 | 18 | 61 | 58-75 |
| ClusterIV-4 | 168 | CCTCTGAGAAGGGTTAT | 61 | 17 | 47 | 7-23 |
| ClusterIV-5 | 169 | GATGCCTTTGTAGCCGGT | 67 | 18 | 55 | 109-126 |
| ClusterIV-6 | 170 | CTGCGTGTTGTCCCGTAT | 67 | 18 | 55 | 52-69 |
| ClusterIV-7 | 171 | TTCTGCAAGGATCCGCGT | 67 | 18 | 55 | 4-21 |
| ClusterIV-8 | 172 | CACCGCGGTGATGATCC | 68 | 17 | 64 | 164-180 |
| ClusterIV-9 | 173 | TCATGGGCATTGACCG | 63 | 16 | 56 | 68-83 |
| ClusterIV-10 | 174 | CCGTCCCTCATGCTGTC | 68 | 17 | 64 | 46-62 |
| ClusterIV-11 | 175 | TTGCCGCCTGTCAGGTT | 66 | 17 | 58 | 10-26 |
| ClusterIV-12 | 176 | CCTCCGCTTTCAACACAT | 64 | 18 | 50 | 114-131 |
| ClusterIV-13 | 177 | GCCTCTCACCATAGAGTG | 67 | 18 | 55 | 14-31 |
| ClusterIV-14 | 178 | CAATATCAAGGATAGTAGGCAATC | 67 | 24 | 37 | 29-52 |
| ClusterIV-15 | 179 | CTTTCCGTGCATTAATGTCC | 66 | 20 | 45 | 8-27 |
| ClusterIV-16 | 180 | AATCCTACGTCCAGCGAG | 67 | 18 | 55 | 13-30 |
| ClusterIV-17 | 181 | CTGTATTTTGTGTCCGCATAC | 66 | 21 | 42 | 13-33 |
| ClusterIV-18 | 182 | ACCGTGGGGATCTTCCTC | 69 | 18 | 61 | 24-41 |
| ClusterIV-19 | 183 | ACTGTGGGAATGTCAGCC | 67 | 18 | 55 | 24-41 |
| ClusterIV-20 | 184 | TAATATCCTGACCCTTTTGC | 64 | 20 | 40 | 57-76 |
| ClusterIV-21 | 185 | AGGTAATCCAGTACCGT | 61 | 17 | 47 | 37-53 |
| ClusterIV-22 | 186 | CCGTGCAACAACAGTTTC | 64 | 18 | 50 | 6-23 |
| ClusterIV-23 | 187 | GCCAAGGCTTTCCAGCAT | 67 | 18 | 55 | 187-204 |
| ClusterIV-24 | 188 | TGACCTCCTGGACGTTATC | 67 | 19 | 52 | 58-76 |
| ClusterIV-25 | 189 | CCGCCCCTTAAATTTGAAG | 65 | 19 | 47 | 5-23 |

^{a.}catcher oligonucleotides have been denoted with the aid of distribution in the clusters. The number of catcher oligonucleotides in the clusters does not represent their importance.
^{b}oligonucleotide sequences shown have reverse complementarity to the relevant sequences of the sense nifH/anfH/vnfH strands. All oligonucleotides are bound to the micro-arrays by the 5′end.
^{c}Tₘ values were calculated using the program MELT 1.1.0 (Jo P. Sanders).
^{d}the position of the oligonucleotides represents the position at which the target sequence is bound [Here, other than as shown in the other figures, the region of the PCR primer (18 nt) has not been included].

### REFERENCES

Beier, M., and D. Hoheisel Jörg. 1999, Versatile Derivatisation of solid support media for covalent bonding on DNA-microchips. Nucleic Acids Res. 27:1970-1977.
Benters, R., C. M. Niemeyer, D. Drutschmann, D. Blohm, and D. Wöhrle. 2002. DNA microarrays with PAMAM dendritic linker systems. Nucleic Acids Res. 30: e10.
Hurek, T., Van Montagu, M., Kellenberger, E., and Reinhold-Hurek, B. (1995) Induction of complex intracytoplasmic membranes related to nitrogen fixation in Azoarcus sp. BH72. Mol Microbiol 18: 225-236.
Hurek, T., Handley, L., Reinhold-Hurek, B., and Piché, Y. (2002) Azoarcus grass endophytes contribute fixed nitrogen to the plant in an unculturable state. Mol Plant-Microb Interact 15: 233-242.
Niemeyer, C., Boldt, L., Ceyhan, B. and Blohm, D. 1999. Evaluation of single-stranded nucleic acids as carriers in the DNA-directed assembly of macromolecules. J. Biomol. Struct. Dyn., 17, 527-538.
Peplies, J., Glöckner, F. O., and Amann, R. 2003. Optimization strategies for DNA microarray-based detection of bacteria with 16S rRNA-targeting oligonucleotide probes. Appl. Environ. Microbiol. 69: 1397-1407.
Southern, E., Mir, K., and Shchepinov, M. 1999. Molecular interactions on microarrays. Nature Genet. Suppl. 21: 5-9.
Zehr, J. P., and McReynolds, L. A. (1989) Use of degenerate oligonucleotides for amplification of the nifH gene from the marine cyanobacterium Trichodesmium thiebautii. Appl Environ Microbiol 55: 2522-2526.
Galloway, J. N., Schlesinger, W. H., Levy, H. I., Michaels, A. F., and Schnoor, J. L. (1995) Nitrogen fixation: Anthropogenic enhancement-environmental response. Global Biogeochem. Cycles 9: 235-252.
Hurek, T., Egener, T., and Reinhold-Hurek, B. (1997) Divergence in nitrogenases of Azoarcus spp., Proteobacteria of the β-subclass. J. Bacteriol. 179: 4172-4178.
Hurek, T., Handley, L., Reinhold-Hurek, B., and Piché, Y. (2002) Azoarcus grass endophytes contribute fixed nitrogen to the plant in an unculturable state. Mol. Plant-Microbe In. 15: 233-242.
Karl, D., Bergman, B., Capone, D., Carpenter, E., Letelier, R., Lipschultz, F. et al. (2002) Dinitrogen fixation in the world's oceans. Biogechem. 57/58.
Tan Z, Hurek T, Reinhold-Hurek B. (2003) Effect of N-fertilization, plant genotype and environmental conditions on nifH gene pools in roots of rice. Environ Microbiol. 5(10):1009-15

1. Method of analyzing samples by means of a ligand binding method with a high phylogenetic resolution in which, through the binding of target sequences of target molecules to catcher sequences of probes, duplexes or complexes are generated and/or duplexes or complexes thus generated are analyzed, wherein the target sequences are partial sequences of the target molecule concerned, and the catcher sequences have a length of 17 mer to 25 mer, and the length of the target molecules is at least four times the length of the target sequence,
wherein within the target sequence, the duplexes or complexes have at least one detectable marking or an accumulation of markings, and at least ten samples are analyzed simultaneously.

2-4. (canceled)

5. Method according to claim 1,
wherein all target molecules have the same number of markings.

6. Method according to claim 1,
wherein at least one hundred samples are analyzed simultaneously.

7. Method according to claim 1,
wherein the marking is a fluorescent marking.

8. Method according to claim 7,
wherein the fluorescent marking is obtained by means of one or more of the following marking agents:
Cy3, Cy5, fluorescein, Texas red, Alexa fluor dyes and other fluorescent dyes.

9-23. (canceled)

24. Method for the determination of catcher sequences, in each case determined for the development of ligand bonds with target sequences each forming a partial sequence of a longer target molecule in such a way that they are complementary to the relevant sections of the target sequences which are to be found in proximity to a marking or an accumulation of markings.

25. Method according to claim 24,
wherein the catcher sequences are determined in such a way that the marking or accumulation of markings is located less than 100 bases from the target sequence or is within the target sequence.

26. Method according to claim 25,
wherein the catcher sequences are calculated by means of a computer program.

27. Set of catcher molecules wherein the catcher molecules have the specificity of that catcher molecule which in each case comprises a sequence selected from SEQ ID NO: 1-189.

28-29. (canceled)

30. Method according to claim 1,
wherein the target molecules are 5′-end-marked.

31. Method of analyzing samples by means of a ligand binding method with a high phylogenetic resolution in which, through the binding of target sequences of target molecules to catcher sequences of probes, duplexes or complexes are generated and/or duplexes or complexes thus generated are analyzed, wherein the target sequences are partial sequences of the target molecule concerned, and the length of the target molecules is at least two times the length of the target sequence, wherein within the target sequence, the duplexes or complexes have at least one detectable marking or an accumulation of markings, and at least ten samples are analyzed simultaneously.

32. Method according to claim 31,
wherein the target molecules are 5′-end-marked.

33. Method according to claim 31, wherein the catcher sequences have a length of up to 35 mer and constituents of the probes of a DNA array, and the length of the target molecules are at least four times the length of the target sequence.

34. Method according to claim 33,
wherein said at least one detectable marking is no further than 0-20 bases distant from the target sequence.

35. Method according to claim 34,
wherein the target molecules are 5′-end-marked.

36. Method according to claim 1 in which target molecules are used having a marking in proximity to or within a target sequence which is a partial sequence of the relevant target molecule, wherein the target molecules are produced by means of a PCR method in which at least one type of dNTP provided with a marking agent is used, and in which the marked dNTPs are incorporated in direct proximity to the target sequence and/or in the target sequence itself, and in which one or more primers is provided with the same or other marking agents.

37. Set according to claim 19,
wherein the catcher molecules are oglionucleotides.

38. Set according to claim 37,
wherein it has at least 10 different catcher sequences.

39. Method according to claim 1, wherein the catcher molecules are at least in part catcher molecules which in each case have the specificity of a catcher molecule comprising a sequence selected from SEQ ID NO: 1-189.

40. Method of analyzing variations of a gene by means of a ligand binding method with a high phylogenetic resolution in which, through the binding of target sequences of target molecules to catcher sequences of probes, duplexes or complexes are generated and/or duplexes or complexes thus generated are analyzed, wherein each target sequence is specific for a certain variation of said gene, and, wherein in the proximity to and/or within the target sequence, the duplexes or complexes have at least one detectable marking or an accumulation of markings.

41. Method according to claim 40, wherein at least ten variations are analyzed simultaneously.
